(19) United States
(12) Patent Application Publication
(10) Pub. No.: US 2025/0281744 A1
(43) Pub. Date: Sep. 11, 2025

(54) ASSIST-AS-NEEDED FUNCTIONAL ELECTRICAL STIMULATION (FES) BASED ON MEASURED USER EFFORT

(71) Applicant: Case Western Reserve University, Cleveland, OH (US)

(72) Inventors: Michael J. Fu, Mayfield Heights, OH (US); Mehmet Akif Gormez, Cleveland, OH (US); Patrick Crago, Cleveland Heights, OH (US); Nathaniel Makowski, Cleveland, OH (US)

(21) Appl. No.: 19/073,427

(22) Filed: Mar. 7, 2025

Related U.S. Application Data
(60) Provisional application No. 63/562,903, filed on Mar. 8, 2024.
(60) Provisional application No. 63/657,150, filed on Jun. 7, 2024.

(51) Int. Cl. A61N 1/36; A61N 1/04

(52) U.S. Cl. CPC A61N 1/36031; A61N 1/0452; A61N 1/36003

(57) ABSTRACT

Functional Electrical Stimulation (FES) can be applied to a user in an assist as needed manner using a closed feedback loop based on user effort. A system can include at least one measuring device to record data indicative of a movement of at least one muscle, at least one stimulating electrode to provide an electrical signal to a muscle, and a controller to implement the closed feedback loop. The controller can instruct the user to try to perform a movement, apply the electrical signal, receive the recorded data, estimate an effort expended by the user, correct the effort expended by the user, determine if the electrical signal should be altered based on at least the user's effort, and if needed, alter the electrical signal. The controller can correct the effort based on a volitional muscle force, a stimulated muscle force, and an occlusion between the two.

### RELATED APPLICATIONS

This application claims the benefit of U.S. Provisional Application No. 63/657,150, entitled “CLOSED LOOP FUNCTIONAL ELECTRICAL STIMULATION (FES) BASED ON MEASURED USER EFFORT,” filed Jun. 7, 2024, and the benefit of U.S. Provisional Application No. 63/562,903, entitled “CLOSED-LOOP FUNCTIONAL ELECTRICAL STIMULATION BASED ON USER EFFORT”, filed Mar. 8, 2024. These applications are hereby incorporated by reference in their entireties for all purposes.

### GOVERNMENT FUNDING

This invention was made with government support under 1942402 awarded by the National Science Foundation. The government has certain rights in the invention.

### TECHNICAL FIELD

This disclosure relates generally to functional electrical stimulation (FES), and more specifically to systems and methods for applying FES in an assist-as-needed manner based on measured user effort taking into account occlusion between volitional muscle force and stimulated muscle force.

### BACKGROUND

Strokes are a leading cause of lifelong disability worldwide. Among many other symptoms, strokes can cause impairment to one or more limbs (otherwise known as paretic limb(s)). Traditional rehabilitation therapy requires daily repetitive use of the paretic limb via structured tasks to improve motor relearning. However, if a patient cannot perform the tasks with the paretic limb, the patient may see no improvement from the rehabilitation therapy. Assistive functional electrical stimulation (FES) systems (and other assistive technologies including robotic systems) have been designed to help the patient perform the tasks with the paretic limb and assist with rehabilitation. Stimulation is applied as the patient performs tasks and then turned off. The stimulation is effort agnostic and can often lead to patients slacking.

During exercises slacking patients rely mostly or entirely on FES to move their paretic limb and do not put in the effort required to properly gain muscle and/or improve neural connections. FES assists muscle movement by creating a stimulation force in a muscle, but this stimulation force can occlude volitional force (e.g., force occlusion) making it harder to tell if a patient is slacking. Additionally, the stimulating electrical signal of FES can interfere with electromyography (EMG) recordings (e.g., EMG occlusion). Slacking can severely limit the effectiveness of rehabilitation and can leave the patient with a permanent disability that could have been at least partially corrected. Current assistive FES systems and other assistive technologies do not account for patient effort to prevent slacking or provide as-needed-assistance.

### SUMMARY

Described herein are systems and methods that provide effective assist-as-needed Functional Electrical Stimulation (FES) that accounts for user effort. A closed-loop feedback loop can account for occlusion between FES created stimulation muscle force and user generated volitional muscle force to accurately determine effort expended by the user at a given task and determine if a user is slacking. The closed-loop feedback loop can then, if necessary, start, stop, or modify the FES to be provided in an assist-as-needed fashion to help the user expending a given amount of effort to complete the given task or to force a slacking user to employ more effort to complete the given task. Thus, the assist-as-needed FES can provide assistance as needed for improved recovery, rehabilitations, or training outcomes, while not allowing a user to slack.

In an aspect, the present disclosure can include a system that includes at least one measuring device, at least one stimulating electrode, and a controller. The at least one measuring device records data indicative of at least one movement of one or more muscles of a user. The at least one stimulating electrode provides an electrical signal to at least a first muscle of the one or more muscles of the user. The electrical signal can be used for FES and can cause a stimulated muscle force. The controller is in communication with the at least one measuring device and the at least one stimulating electrode and includes a non-transitory memory to store instructions and a processor to execute the instructions to cause the following actions: output a command to the user to perform or attempt to perform the at least one movement of the one or more muscles (e.g., a given task); and in response to the instruction, the user performs or attempts to perform the at least one movement of the one or more muscles, which creates a volitional muscle force. Then, the electrical signal having at least one parameter can be applied, via the at least one stimulating electrode, to the at least the first muscle of the one or more muscles during the user performing or attempting to perform the at least one movement of the one or more muscles. Data indicative of the at least one movement the one or more muscles over a time period of constant muscle contraction can be received from the at least one measuring device. The data indicative of the at least one movement of the one or more muscles includes both the stimulated muscle force and the volitional muscle force. An effort expended by the user during the at least one movement of the one or more muscles over the time period can be estimated. The effort expended by the user for at least occlusion between the volitional muscle force and the stimulated muscle force can be corrected and it can be determined whether the electrical signal should be altered based on the corrected effort expended by the user and a movement goal, where if the electrical signal should be altered then the at least one parameter of the electrical signal is changed, and if the electrical signal should not be altered, then make no change to the electrical signal.

In another aspect, the present disclosure can include a method for estimating effort of a user of assisted functional electrical stimulation (FES) executed by a system comprising a processor. Electromyography (EMG) signals can be received for a time period of a constant muscle contraction of a movement, wherein the movement is at least partially driven by volitional movement and at least partially driven by stimulated movement from the FES. Effort expended by the user performing or attempting to perform the movement, an occlusion factor for the amount of occlusion caused by the volitional movement and the stimulated movement simultaneously, and a fatigue factor for the movement can be estimated. A corrected effort can then be determined based on the estimated effort, the occlusion factor, and the fatigue factor.

In a further aspect, the present disclosure can include a method for controlling effort dependent contralaterally controlled functional electrical stimulation (CCFES) rehabilitation. A system comprising a processor can execute the method. A user can be instructed to perform a movement, where the user will at least attempt to perform the movement. A stimulation can be applied by the system via at one or more electrodes to a muscle activated by the movement. The stimulation can include a FES signal. A corrected effort percentage for the movement can then be determined that accounts for user expended effort, an occlusion factor, and a fatigue factor; a task tracking error representative of the difference between a movement goal and the movement performed by the user; a movement error representative of a difference in a measurement of the movement normalized to a measurement of the movement due to the stimulation; and at least one parameter of the stimulation to modulate based on the corrected effort percentage, the task tracking error, and the movement error, wherein the at least one parameter of the stimulation controls stimulation intensity. The at least one modulated parameter can be applied to a stimulator in communication with the one or more electrodes to modulate the stimulation.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram of a system that can provide effort dependent assistive stimulation of one or more muscles of a user;

FIG. 2 is a more detailed diagram of the system of FIG. 1;

FIG. 3 is a diagram of a controller of the system of FIG. 1;

FIGS. 4 and 5 show the control of effort dependent assistive electrical stimulation implemented by the system of FIG. 1;

FIG. 6-9 are process flow diagrams illustrating methods for performing effort dependent assistive FES;

FIG. 10 is a conceptual illustration of an experimental methodology;

FIG. 11 is a diagram of an experimental set up;

FIG. 12 shows a graphical representation of experiment target profiles divided into segments;

FIG. 13 shows a graphical representation of example time instances of visual feedback shown to participants during an experimental trial;

FIGS. 14 and 15 show graphical representations of experimental calculations for correcting effort;

FIG. 16 shows an illustration of an experimental setup and a graphical representation of data generated in that experiment;

FIG. 17 shows a control diagram illustration of a scheme for effort dependent contralaterally controlled FES;

FIGS. 18-20 show graphical representations of experimental results and data manipulation;

FIG. 21 shows a control diagram illustration of another scheme for effort dependent contralaterally controlled FES; and

FIG. 22 shows a graphical representation of experimental results.

### DETAILED DESCRIPTION

### I. DEFINITIONS

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “paretic” can refer to at least a portion of a person's body afflicted with muscular weakness caused by nerve damage or disease (e.g., at least partial paralysis otherwise called paresis). For example, a stroke can cause a patient to have one or more paretic limbs.

As used herein “electrical signal” can refer to a function (waveform) that conveys information about the behavior or attributes of an electrical phenomenon, such as electric current or voltage, that varies with time and/or space. For example, the electrical signal can be an alternating current signal and/or a direct current signal. An electrical signal can have one or more variable parameters, including but not limited to voltage, pulse width, duty cycle, frequency, current, amplitude, and waveform change.

As used herein, “Functional Electrical Stimulation” or “FES” can refer to a technique that applies an electrical signal to one or more muscles to generate muscle contraction(s). For example, FES can be used with patients suffering from a spinal cord injury, a head injury, a stroke, a neurological disorder, or the like. In another example, FES can be used for rehabilitation of a patient with a muscular injury and/or training/strengthening one or more muscles of an injured and/or a non-injured user. As a non-limiting example, a muscle such as the first dorsal interosseous can be stimulated by FES. As another non-limiting example the FES can include a current controlled, bipolar pulse wave modulation (PWM) stimulation configured with at least a voltage ranging from about 20 V to about 150 V, depending on an impedance of the muscle, skin, etc.; a fixed current value (e.g. fixed per subject) that can be set between 25 mA and 35 mA; and a pulse width that can be varied from two or more values from 0 microseconds to 255 microseconds (e.g., to vary stimulation intensity).

As used herein, “movement” can refer to a muscular movement to change a position and or location of one or more portions of a user's body. A muscular movement can include a muscle contraction (causing a measurable muscle force) in at least one muscle of the user's body and a relaxation in at least one other muscle of user's body in response. Common movement pairs can include, but are not limited to: flexion and extension, abduction and adduction, pronation and supination, elevation and depression, protraction and retraction, inversion and eversion, and the like

As used herein, “stimulated muscle force” can refer to a muscle force caused by an electrical stimulation (e.g., FES) evoking an action potential that causes muscle contraction.

As used herein “volitional muscle force” can refer to a muscle force caused by a user intentionally evoking an action potential to cause at least one muscle contraction.

As used herein, the term “slacking” can refer to an intentional or unintentional decrease or reduction in the intensity of volitional muscle force, particularly when a patient relies too heavily (e.g., above a predetermined threshold) or solely on an assistive therapy (e.g., FES) to generate movement. For example, a patient can be regarded as slacking when the patient's volitional muscle force is intentionally too low to cause a complete movement and the stimulating muscle force causes the movement.

As used herein, the term “effort” can refer to a percentage of a maximum force possible for a patient to exert. Effort can be measured and/or estimated, for example using EMG recordings. In some instances, effort can be expressed as a percentage: current effort (100% or less effort) compared to total possible effort (100% effort). User expended effort can include both stimulating muscle force and volitional muscle force. As used herein, estimated effort can refer to mean absolute value of estimated volitional force signal (extracted from an EMG signal) over mean absolute value of the maximum voluntary contraction. However, it should be understood that this is not intended to be limiting and effort can be measured and/or estimated using other features of an EMG signal, such as, but not limited to, slope sign changes, signal length, zero crossings, and the like. As used herein corrected effort can refer to the occlusion and/or fatigue-factor corrected user expended effort. Corrected effort can be defined as the estimated effort plus the occlusion factor corrected user expended effort and/or the fatigue factor corrected user expended effort.

As used herein, the term “fatigue” can refer to an involuntary decrease in maximal force or power production in response to muscle contraction. For example, fatigue can decrease a total force/power production possible in response to the muscle contraction. Fatigue is generally exercise induced and can increase to the point where muscles are exhausted and cannot complete a task (e.g., an instructed movement). It should be noted that slacking and fatigue are not the same, fatigue is involuntary.

As used herein, the term “occlusion” can refer to the phenomenon when two or more signals react together to reduce the sum (superposition) of the effect of each of the signals individually. Occlusion can include at least force occlusion and EMG occlusion. For example, stimulation-evoked action potentials and voluntary action potentials occlude one another long the motor axon using mechanisms, such as refractory block, collision, and resetting, to reduce the number of both voluntary and stimulation-induced action potentials that reach the muscle end plate. In another example, the stimulation electrical signal can at least partially occlude the measuring EMG signal.

As used herein, the terms “patient”, “user”, “subject”, or the like can be interchangeable and can refer to any warm-blooded organism that can have the systems and/or methods used on them for rehabilitation and/or training purposes. Generally, the terms as used herein describe a human patient, but the systems, methods, and techniques described herein can also be used with respect to other warm-blood organisms (with any necessary small modifications).

### II. OVERVIEW

Strokes, injuries, and many other conditions can negatively affect a person's ability to move one or more muscles of one or more limbs causing the one or more limbs to be paretic limbs. Rehabilitation can allow a patient suffering from stroke, injury, or many other conditions to work the one or more muscles and, hopefully, eventually regain the ability to move the one or more muscles of the one or more paretic limbs. Traditional rehabilitation therapy requires daily repetitive use of the one or more muscles via structured tasks to improve motor relearning and/or muscle structure and function. However, if the patient cannot perform the tasks, the patient may see no improvement from the rehabilitation therapy alone. Similarly, muscle training can improve muscle strength in an athlete and/or healthy person during a workout or training session (in such cases the paretic limb can be any limb being worked on by an exercise).

Different assistive technologies and rehabilitation/training devices (e.g., bands, resistive devices, exercise equipment, etc.) have been developed to help patients recover and athletes train. One example that is particularly useful for stroke patients with paretic limbs (but not limited to use with stroke patients) is assistive functional electrical stimulation (FES). Systems used for assistive FES have been designed to provide an electrical stimulation of FES to a patient to help the patient perform the tasks with the paretic limb and assist with rehabilitation/training. However, patients may slack during exercises, relying mostly or entirely on the FES to move the paretic limb. Slacking can severely limit the effectiveness of rehabilitation/training and can leave the patient with a permanent disability that could have been at least partially corrected. Moreover, user effort/slacking is not accurately determinable because the stimulation force (created by the electrical stimulation of FES) occludes volitional force (e.g., force occlusion) and the stimulating electrical signal of FES can interfere with electromyography (EMG) recordings (e.g., EMG occlusion). Current FES systems agnostically provide stimulation and do not account for patient effort to prevent slacking, increase effort, and/or increase active patient participation. This is at least in part because these current assistive FES systems cannot effectively determine patient effort. Without feedback of patient effort (e.g., volitional movement), FES systems cannot distinguish between active participation and passive reliance on stimulation. This lack of effort monitoring creates a gap where participants may not fully engage in synchronized tasks, such as simultaneously opening and closing both hands during bilateral practice. The resulting inconsistent or passive movements reduce the therapeutic benefit by limiting active motor engagement, which is critical for neuroplastic changes and motor relearning. Additionally, without an effort-dependent feedback mechanism, participants may not be sufficiently challenged to exert enough effort to achieve their full potential, further undermining the overall efficacy of therapy and diminishing long-term functional outcomes.

Described herein are systems and methods that provide assist-as-needed FES that accounts for user effort, including determining if the user is slacking. The assist-as needed FES includes a closed-loop feedback loop that accurately determines effort expended by the user at a given task and determine if a user is slacking and then provides as needed stimulation. The user effort can be accurately determined by accounting for occlusion between the FES stimulated muscle force and the volitional muscle force. Additionally, the systems and methods can tell the difference between fatigue and slacking and can adjust the FES accordingly to assist the user as needed to provide a user with improved rehabilitation and/or training. In some instances, the systems and methods can also address the gap between what a patient thinks they are doing and what they are actually accomplishing by incorporating monitoring and adaptive feedback to enhance efficacy of treatments.

### III. SYSTEMS

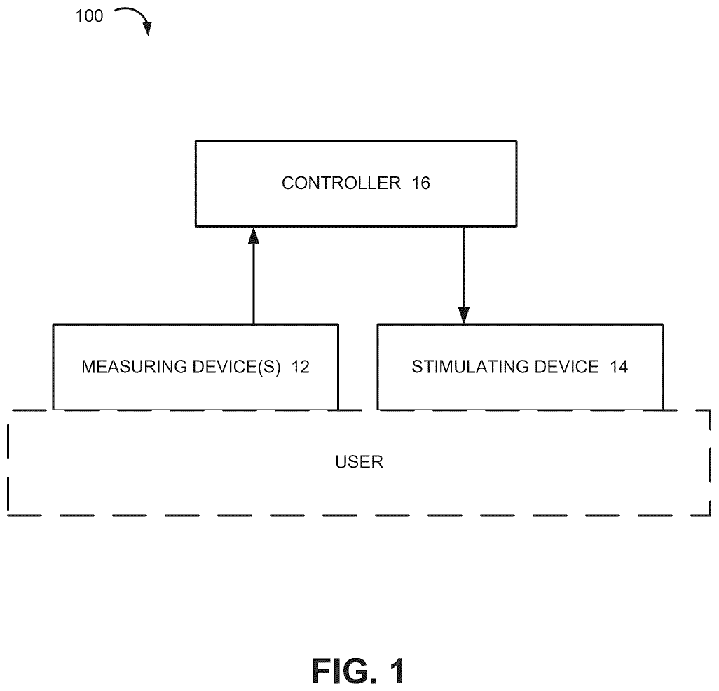

Shown in FIG. 1 is an assist-as-needed Functional Electrical Stimulation (FES) system 100 (also referred to as closed loop, effort dependent FES or the like) that can be used as part of a rehabilitation program for one or more neurological and/or muscular conditions, a physical training program for a user to improve muscle condition, as part of one or more video game systems, or the like. The system 100 can particularly be used to prevent and/or reduce slacking in a user undergoing rehabilitation. For example, the user can suffer from epilepsy and/or may have suffered a stroke and can have one or more paretic limbs or a paretic and non-paretic side. In other example, the user can have a neurological condition affecting the one or more muscles, be an athlete in training, simply be training to improve performance of the one or more muscles, or the like. The term “assist-as-needed” when used to describe the FES system 100 can generally refer to a form of FES that utilizes an effort dependent feedback loop to determine whether a user is at least one of slacking at a task, making an appropriate effort but not succeeding at the task, or making an appropriate effort and succeeding at the task, then determine an amount(s) and/or location(s) for applying FES, and then applying the FES at the determined amount(s) and/or location(s) can be applied at a given level to help the user succeed at the task. Based on the determinations of user effort the FES can be started, stopped, increased, decreased, left at a current amount, change location, or the like. For instance, if the user is determined to be slacking than the FES can be not applied, decreased, or turned off. The assist-as-needed FES systems, and methods, are described in greater detail below.

The system 100 can apply a stimulation to one or more of a user's muscles to assist the user in moving the one or more muscles based on an effort expended by the user. The system 100 can include at least one measuring device (e.g., measuring device(s) 12), a stimulating device 14, and a controller 16. It should be understood that, in some instances, two or more of the measuring devices 12, the stimulating device 14, and the controller 16 can be within a common housing. The measuring device(s) 12 can record data indicative of at least one movement of one or more muscles of the user and send the data to the controller 16. The stimulating device 14 can provide a stimulation to at least one of the one or more muscles of the user. The controller 16 can be in communication (wired and/or wireless) with the measuring device(s) 12 and the stimulating device 14 to receive the data from the measuring device(s) and then send at least one parameter to the stimulating device 14.

The measuring device(s) 12 can record data indicative of at least one movement of one or more muscles of the user. The at least one movement can be, for example, a rehabilitation or training exercise. The data can be electrical data, force data, motion data, or the like. The measuring device(s) 12 can be, for example, at least one of: at least one electromyography (EMG) electrode, at least one force sensor, at least one bend sensor, at least one inertial sensor, at least one motion capture system (e.g., detection unit and at least one marker), at least one rehabilitation device (e.g., resistance band, exercise machine, or the like), etc. The measuring device(s) 12 can be at least partially in contact with a portion of the user's body (e.g., position on the user's skin, in contact with a muscle, or the like) relative to the muscle(s) being targeted for the stimulation (e.g., the muscle(s) being targeted for stimulation, an antagonist of the muscle(s) being targeted for stimulation, a helper muscle for the muscle(s) being targeted for stimulation, or the like).

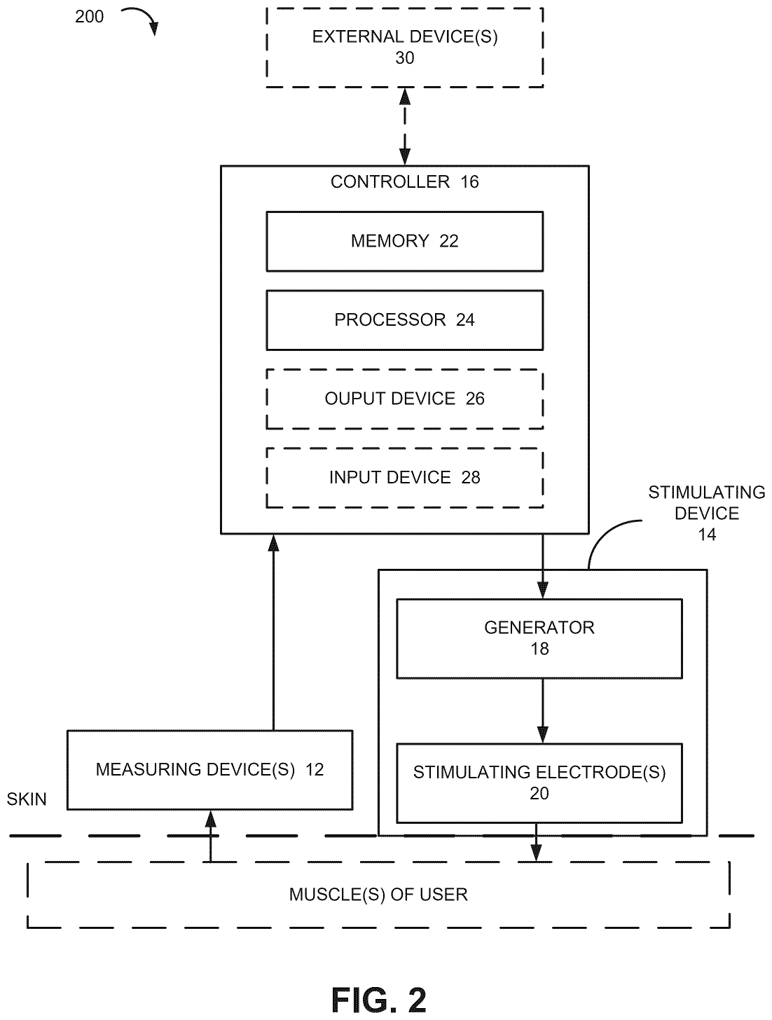

The stimulating device 14 can provide at least electrical stimulation (configured by controller 16) to the user (e.g., muscle(s) of the user, nerves connected to muscle(s) of the user, or the like) to cause muscle contraction in the muscle(s). For instance, the simulating device 14 (e.g., shown in FIG. 2) can include a generator 18 connected to at least one stimulating electrode (stimulating electrode(s) 20) to provide an electrical signal, which may be configured by the controller 16 based on the data from the measuring device(s) 12, to at least a first muscle of the one or more muscles of a user. The electrical stimulation can be used for functional electrical stimulation (FES) for rehabilitation of a limb comprising the one or more muscles. The stimulating device 14 can, in some instances, alternatively and/or additionally include at least one magnet that can modulate one or more magnetic fields to stimulate the one or more muscles of the user. If the stimulating device 14 includes both electrical and magnetic stimulation components, then the electrical stimulation components and the magnetic stimulation components can stimulate the same or different muscles. In some instance, the stimulating device 14 and the measuring device(s) 12 can be positioned on the same muscle(s) and/or on the skin over the same muscle(s).

As noted, FIG. 2 shows how the stimulating device 14 can include a generator 18 and at least one stimulating electrode (e.g., stimulating electrode(s)) 20 that can apply an electrical signal having at least one parameter to the one or more muscles of the user. The generator 18 can provide the electrical signal to one or more of the stimulating electrode(s) 20 and the one or more of the stimulating electrode(s) can apply the electrical signal. It should be understood that only a single electrical signal is described herein for ease of illustration, but can be more than one electrical signal, where each of the stimulating electrode(s) 20 applies a same and/or different electrical signal depending on the prescription and/or instruction for stimulation. The at least one parameter of the electrical signal(s) can include, for example, voltage, pulse width, duty cycle, frequency, current, amplitude, and/or waveform change, or the like.

The controller 16 can be in communication, wired and/or wireless with the measuring device(s) 12 and the stimulating device 14, and, optionally, one or more external devices (e.g., external device(s) 30). The controller 16 can include a non-transitory memory (e.g., memory 22) that can store instructions and data and a processor 24 that can at least execute the instructions. The controller 16 can, optionally, include an output device 26 and/or an input device 28 for communication to and/or from the controller 16. For example, the output device 26 can be a visual display (e.g., a visual screen, an indicator light, or the like), a speaker, a tactile and/or haptic feedback device, or the like that can communicate an instruction, warning, and/or other indication to a user. The output device 26 can convey instructions to perform or attempt to perform the movement of the one or more muscles to the user, if the user needs to rest, if the user should try harder, if the user is doing well, alert the user if the effort expended by the user is below a predetermined threshold, or the like. The input device 28 can be a user interface such as a touch screen, keyboard, computer mouse, buttons, microphone, or the like for a user and/or a medical professional to input information to the controller 16. The controller 16 additionally can include any other circuitry and/or components for general use such as power components (e.g., battery, wall power components, etc.), circuitry, wireless transceivers, exterior housing components, or the like.

The external device(s) 30 can also, additionally and/or alternatively, function as the output device 26 and/or the input device 28 and can be in bi-directional communication with the controller 16. The external device(s) 30 can include, for example, a computer, tablet, television, mobile device, and/or video gaming system. The external device 30 may be used to update one or more prescriptions, instructions, and/or rules for the controller 16 applying the user effort dependent assistive FES, if for example, the external device 30 belongs to a medical, rehabilitation, or training professional. The external device(s) 30 can alternatively and/or additionally include training or exercise equipment (devices, weights, resistive bands, etc.) that may include one or more sensors and/or processors to communicate with the controller 16 to further facilitate the user effort dependent assistive FES (e.g., provide additional physiological and/or movement information about the user at a given time).

As shown in FIG. 2, the stimulating device 14 can be in direct contact with the skin of the user over the one or more muscle(s) to be stimulated. It should be noted that the stimulating electrode(s) 20 specifically can be in contact with the skin (e.g., can be held in place with a skin safe adhesive, a bandage, or the like) and the generator 18 can be remote but in electrical communication with the stimulating electrode(s) 20 to provide the electrical signal(s). The generator 18 and the stimulating electrode(s) 20 can be embodied in a single stimulating device 14 (as shown) or can be embodied as separate but electrically connected devices. Additionally, as previously noted, while shown separately any portions of the controller 16, the stimulating device 14, and the measuring device(s) 12 may be embodied within one or more housings (e.g., the controller and the stimulating device may be combined in a single device, the controller and the measuring device(s) can be combined in a single device, all three can be in a single device, or the like). Generally, the measuring device(s) 12 can be at least partially attached to the skin of the user (e.g., by a skin safe adhesive, a bandage, or the like) (e.g., EMG electrodes, force sensor(s), bend sensor(s), inertial sensor(s), motion capture marker(s), or the like) and/or can include one or more components separate from the user (e.g., a camera of a motion capture system, a separate rehabilitation device, or the like), for instance least one electromyography EMG electrode, at least one force sensor, at least one bend sensor, at least one inertial sensor, at least one motion capture system, and/or at least one rehabilitation device. It should be further noted that in some circumstances (not shown) the measuring device(s) 12 and/or at least a portion of the stimulating device 14 can be implanted subcutaneously.

To provide effort dependent assistive FES to the user the controller 16 can instruct the stimulating device 14 to provide a stimulation (e.g., to cause a muscle contraction in the one or more muscles of the user). For instance the controller 16 can provide at least one parameter of an electrical signal to the generator 18 (e.g., the initial setting can be stored in memory based on population data and/or user specific data) and the generator can generate and provide the electrical signal to one or more of the stimulating electrode(s) 20 to apply the electrical signal to the one or more muscles. The user can be simultaneously attempting a movement that includes use of the one or more muscles (e.g., based on instructions from the controller 16). The application of the electrical signal can facilitate the user successfully performing the movement. It should be noted that the electrical signal can be applied to cause a contraction in a single muscle used for a movement or multiple muscles used for a movement. The measuring device(s) 12 can measure data indicative of the movement of the one or more muscles (e.g., electrical signals, force signals, or the like) and feed this data back to the controller 16.

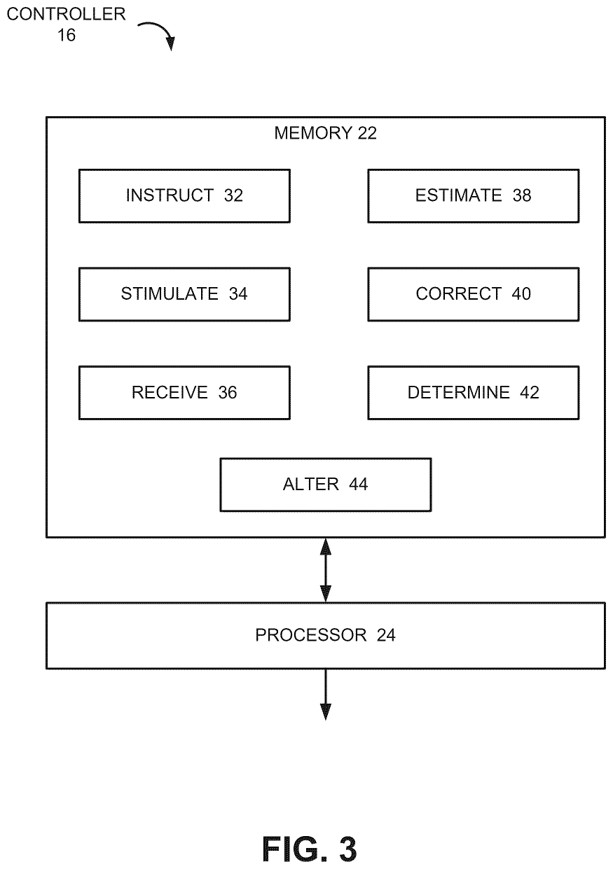

An example of the controller 16 and the associated functionality to apply assist-as-needed FES are shown in further detail in FIG. 3. The non-transitory memory (e.g., memory 22) can store instructions and data and a processor 24 that can communicate with the memory 22 and at least execute the stored instructions to affect the rest of the system (e.g., system 100, 200, or the like). An output can include an instruction or command (instruct 32) the user to perform or attempt to perform the at least one movement of the one or more muscles. This instruction can be, for instance, a verbal command, a visual of the movement, part of a movie and/or video game, or the like). The instructions may include a number of times the at least one movement should be performed, a weight value, a time period to complete and/or hold portions of the movement (e.g., X seconds lift, Y seconds hold, Z seconds lower, or the like), a combination/pattern of movements to perform, and/or the like. In response to the instruction, the user can perform, or at least attempt to perform if the movement cannot be completed, the at least one movement of the one or more muscles. The performing or attempting to perform the at least one movement can create muscle contraction(s) that create a volitional muscle force in the one or more muscles.

The controller 16 can instruct the stimulating device (e.g., stimulating device 14) to stimulate 34 the one or more muscles of the user by applying (e.g., via the at least one stimulating electrode 20) the electrical signal having at least one parameter to the at least a first muscle (of the one or more muscles) during the user performing or attempting to perform the at least one movement of the one or more muscles. The application of the electrical signal can cause a stimulated muscle force in the one or more muscles that is not created by the user themselves. The data indicative of the at least one movement of the one or muscles (including the at least he first muscle) can be recorded by the measuring device(s) (e.g., measuring device(s) 12) can received 36 by the controller. The data indicative of the at least one movement of the one or more muscles can be for a time period of constant muscle contraction during the at least one movement. The data indicative of the at least one movement of the one or more muscles can include both the volitional muscle force (created by the user) and the stimulated muscle force (created by the electrical stimulation).

After receiving (receive 36) the data indicate of the at least on movement of the one or more muscles, which includes both the volitional muscle force and the stimulated muscle force, the instructions can then make determinations about the user's level of effort during the at least one movement (e.g., to prevent slacking, to improve muscle use/movement, or the like). The controller 16 can estimate 38 an effort expended by the user during the at least one movement of the one or more muscles over the time period (of constant muscle contraction) and correct 40 the effort expended by the user for at least occlusion between the volitional muscle force and the stimulated muscle force. The effort expended by the user can be further corrected with a fatigue correction factor to account for muscle fatigue. For instance, the fatigue correction factor can be based on a function of the median frequency of the data received from the measuring device(s).

The controller 16 can then determine 42 whether the electrical signal should be altered 44 based on the corrected effort expended by the user, a movement goal, and/or a tracking error (e.g., stored in the memory 22 and set by the user and/or a medical, training, or rehabilitation professional). The determination can further include the proximity of the user's performed (or attempted) movement to a movement goal (e.g., determined based on at least the data indicative of the at least one movement compared to expected values for the completed correct at least one movement). If the electrical signal should be altered, then the controller can change (alter 44) the at least one parameter of the electrical signal (e.g., send the changed at least one parameter to the generator 18). For example, if the user is close to the movement goal and/or the corrected effort expended by the user is below a predetermined threshold, then the electrical stimulation can be increased to give the user additional help. If the user is not close to the movement goal and/or the corrected effort expended by the user is above another predetermined threshold, then the electrical stimulation can be decreased to force the user to work harder. If the electrical signal should not be altered, then no change can be made to the at least one parameter of the electrical signal. In addition to determining if the at least one parameter of the electrical signal should be changed the controller can also determine if the user is slacking, fatigued, doing the movement correctly, or the like and output an indication thereof (e.g., at least one of visual, audio, haptic alert, or the like).

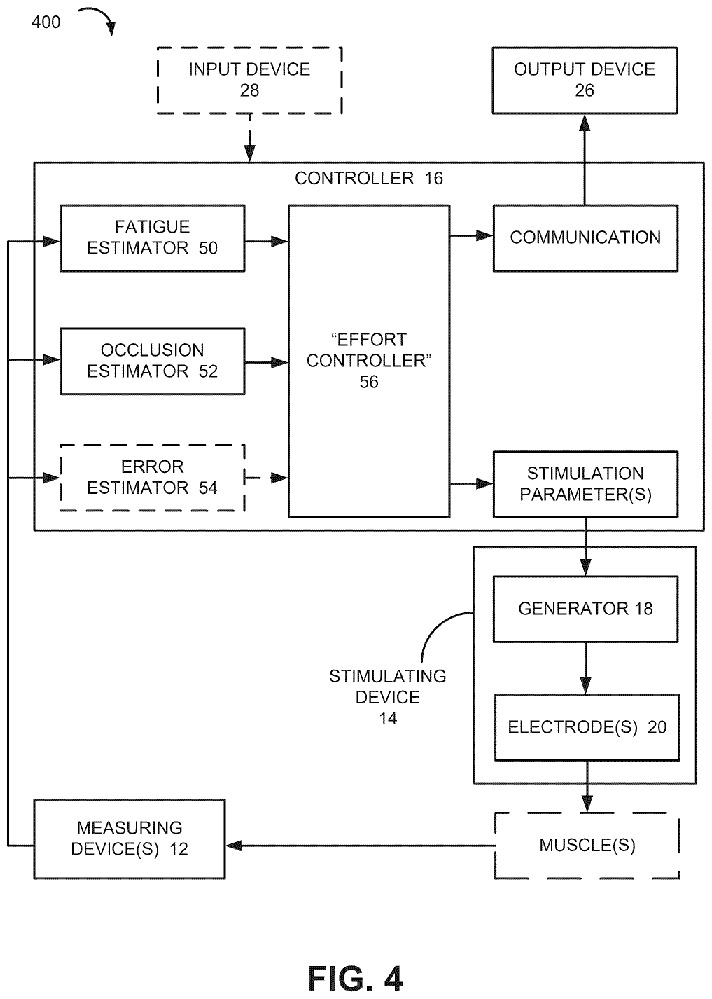

FIG. 4 shows an example control diagram 400 for the system (e.g., 100, 200, or the like) performing effort dependent assistive FES that includes estimating the effort of the user and “effort correction”. As previously discussed, the controller 16 can instruct a user to perform, or attempt to perform, at least one movement of one or more muscles and through stimulating device 14 provide an electrical signal (e.g., FES) to the user to cause further muscle forces of the one or more muscles. The measuring device 12 can record the data indicative of the at least one movement (e.g., electrical data, force data, or the like). The data indicative of the at least one movement can include a volitional force measurement portion and a stimulation measurement portion. EMG signals will be described herein for ease of illustration, but it should be understood that alternative or additional other data types could be used in place of the EMG signals.

The controller 16 can receive the EMG signals for a time period of the at least one movement. For instance, the time period can be for a constant muscle contraction of the at least one movement. Because the user is both performing (or attempting to perform) the at least one movement and the stimulation is being applied, the at least one movement is at least partially driven by volitional movement and at least partially driven by stimulated movement from the stimulation (e.g., FES). The controller can include a fatigue estimator 50 and an occlusion estimator 52, and optionally an error estimator 54, into which the EMG signals (or data derived from the EMG signals) can be fed. In some instances, prior to use for calculations the EMG signals can be filtered, for example to remove stimulation elicited motor response artifacts, and an extracted feature of the filtered EMG signals can be determined (for use in the calculations) (e.g., mean absolute value, sign changes, signal length, zero crossings, etc.).

The fatigue estimator 50 can estimate a fatigue factor for the at least one movement (e.g., based on how fatigued the one or more muscles of the user are). The fatigue factor can be estimated based on a function of the median frequency of the filtered EMG signal over the time period. The occlusion estimator 52 can estimate an occlusion factor for the amount of occlusion between the volitional muscle force and the stimulated muscle forces caused by the simultaneous volitional and stimulated movement. The occlusion factor can be estimated by at least partially linearly regressing the volitional movement and stimulated movement of the user at a time in the time period. The error estimator 54 can estimate an error factor that can account for at least one of tracking error, error in the measurements, drift, and the like. In some instances, additional measurement data from one or more non-paretic muscles (e.g., same muscle(s) on the other side of the body) can be used by the error estimator 54 for error tracking. As an example, the tracking error can be a factor of how close the user's movement is to the movement goal. The effort controller 56 can estimate the effort expended by the user performing or attempting to perform the movement and can correct the effort of the user based on the estimated effort, the occlusion factor, the fatigue factor, and optionally the error factor. The estimate of the effort expended by the user perform or preforming or attempting to perform the movement can be estimated dividing the mean absolute value of the filtered EMG signals for the time period by a maximum mean absolute value of contraction of the muscle.

After determining the corrected effort the controller 16 can send a communication based on the corrected error to an output device 26. The communication can include, for instance, an instruction or indication for the user to push harder, that the user is doing well, that the user is fatigued and should stop, or the like. The controller 16 can additionally and/or alternatively modulate the at least one parameter of the electrical stimulation in response to the corrected effort determination and send the modulated at least one parameter back to the stimulating device 14. For instance, the intensity of the stimulation can be increased when the corrected effort is above a threshold and/or the tracking error is below threshold in indicating the user is close to the movement goal. In another instance the intensity of the stimulation can be lowered or kept the same when the corrected effort is below threshold and/or the tracking error is above another threshold indicating the user is far from the movement goal. If the user is fatigued the controller 16 can stop the stimulation and instruct the user to rest when the corrected effort indicates fatigue is above a threshold (e.g., compared to a maximum corrected effort previously determined for the user).

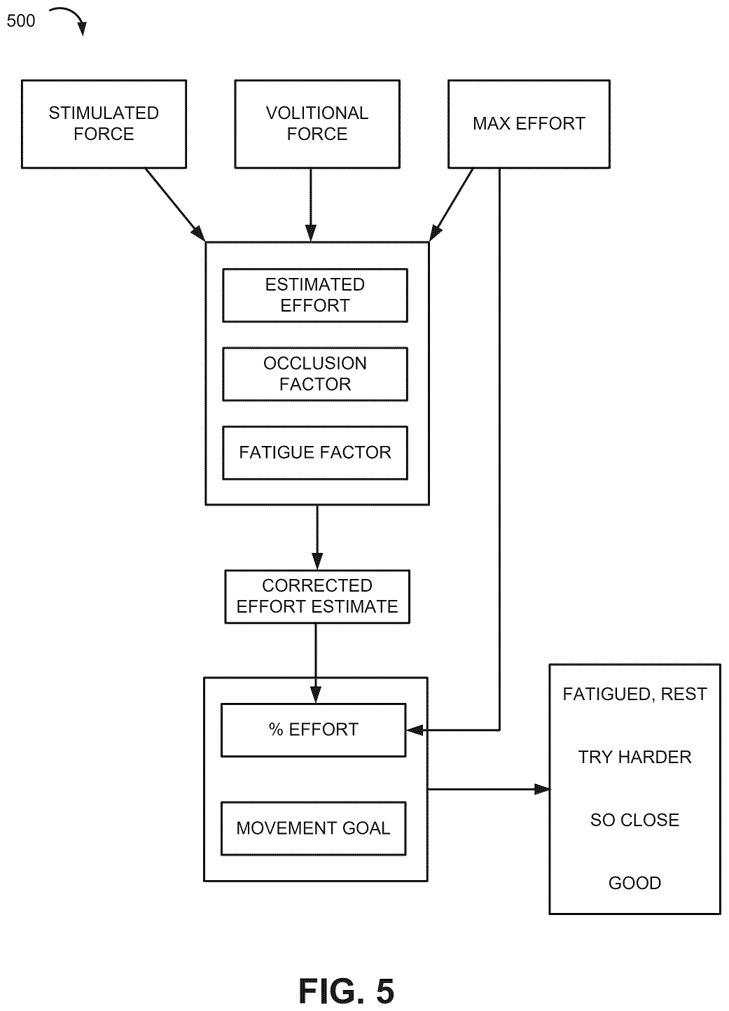

FIG. 5 shows a diagram 500 of an example of how data is transformed through the control loop of FIG. 4. The stimulated force and the volitional force can be measured (e.g., from the one or more muscles) at a time. It should be noted that while shown as separate values, these forces occlude each other and cannot be measured separately when stimulation is being applied to the volitionally moved muscle. The maximum effort can be measured from the user (e.g., from a non-paretic side, a pre-injury/disease/training recording, or the like of the at least one movement at full force) and/or can be determined for the user (e.g., based on user and/or population type specific data) and stored in the memory.

The estimated effort at the time can be determined based on the stimulated force, the volitional force, and the max effort data. The estimated effort can be determined by Eₑ=MAVₑ/MAVₘₐₓ, where MAVₑ is the mean absolute value of the estimated vEMG signal after m-Waves are filtered and MAVₘₐₓ is the MAV during MVC. More generally, the uncorrected effort estimate generally, Eₑ, can be calculated as the rate of an EMG feature divided by the EMG feature's value at maximum voluntary contractions, after m Wave removal (e.g., as written above Eₑ=MAVₑ/MAVₘₐₓ). It should be understood that other EMG features such as slope sign changes, signal length, etc. can be used alternatively.

The occlusion factor and fatigue factors can then be determined. The occlusion factor (Ê_{O}(V,S)) includes components of both the volitional and the stimulated forces, accordingly volitional effort and stimulated effort need to be calculated. A volitional effort (V) of the user can be determined based on V=Fᵥ/Fₘₐₓ where Fᵥ is volitional force and Fₘₐₓ is the force during maximum voluntary contraction (MVC). A stimulated effort (otherwise known as stimulated muscle activation intensity) can be determined based on (S=Fₛ/Fₘₐₓ) normalized to Fₘₐₓ in units of % Effort, where Fₛ is stimulated force. The occlusion correction factor estimation model can be given as Ê_{O}(V,S)=a+bV+cS where, a, b, and c are the linear fitting coefficients. The occluded effort can be determined based on a linear regression (Ê_{O}(V,S)) of experimental results of a similar user and/or calibration results for a given user at different combinations of voluntary effort and stimulation intensities. The fatigue factor can be determined by a function of the volitional force (e.g., from the EMG signals) median frequency f compared to the maximum volitional force. In other words, the fatigue factor (the muscle fatigue) can be estimated as a proportional function of mean and/or median frequency shifts in the frequency spectrum of an EMG recording for a give movement. For instance, if the mean and/or median frequencies shift an average of 10% then that can be an indication of 10% fatigue.

The corrected effort estimation can be corrected for occlusion, and optionally for fatigue as well. An occlusion corrected effort estimation can be defined by E=Eₑ+Ê_{O}(V,S) and the occlusion and fatigue corrected effort estimation can be defined by E=Eₑ+Ê_{O}(V,S)+V_{fatigue}(f). The corrected effort estimation can be a more accurate measure of the effort the user is putting into a movement because it accounts for the occlusion between the stimulation signal and the user's own voluntary movement, and optionally the fatigue of the user.

Each of the corrected effort estimations can be determined as a percentage compared to 100% voluntary effort (which can be pre-determined based on the max effort). The percent effort based on the corrected effort estimation (corrected for occlusion or corrected for occlusion and fatigue) can then be compared to predetermined thresholds and the movement (or attempt) with a movement goal to determine if at least one parameter of the stimulation should be altered and/or a communication to be sent to the user (e.g., fatigued, rest; try harder and so close (encouragement); good job (validation), or the like). The movement goal for the at least one movement can be received (e.g., from an external device 30 or input device 28) or located (e.g., in memory 22). The movement goal can be determined specifically for the user or for a population to which the user belongs. The data indicative of the at least one movement and the corrected effort expended by the user (corrected for occlusion and/or fatigue) can be compared with the movement goal and effort thresholds that can be predetermined, can be updated as the user improves, or the like.

For instance, if the movement is hand opening then the assistance needed for assist as needed FES can be determined based on:

$$Stim ( \% ) = \{ \begin{matrix}R ( {θ}_{non - paretic} \cdot Effort , fatigue < d and \vert {Error}_{tracking} \vert \geq c \\ MG \cdot Effort \cdot \frac{\vert {Effort}_{tracking} \vert}{c} , fatigue < d and \vert {Error}_{tracking} \vert < c \\ 0 , fatigue \geq d and \vert {Error}_{hand} \vert \geq m and Effort > k\end{matrix}$$

Where the stim (%) can be any at least one parameter of the stimulation that relates to stimulation intensity (e.g., pulse width or the like) and can range from 0% (no stimulation) to 100% (the highest level of stimulation is applied). R is a recruitment curve function converting the degree of non-paretic hand opening θ_{non-paretic} to stimulation intensity ranging from 0 to 100%. It should be noted that θ_{non-paretic} can be replaced by any other representation(s) of the movement goal, which can be in terms of degrees, EMG, body pose, inertial measurement, or the like depending on the movement. Effort ranges from 0 (zero effort) to 1.0 (maximum (100%) effort and is based on the percent corrected effort. Fatigue can be the amount of median frequency shift (in Hz) of the M-wave from the EMG signals and d is a constant number. Error_{tracking} is a value between 0 (lowest error) and 1.0 (highest error) representing the different between the movement and the movement goal, and c is a constant representing an error threshold ranging from 0 to 1. Error_{hand} is a value between 0 (lowest error) and 1.0 (highest error) representing the different from the movement and the movement goal normalized for the stimulated force and m is a constant number. The constants can be tuned to each user during a calibration and/or setup session.

### IV. METHODS

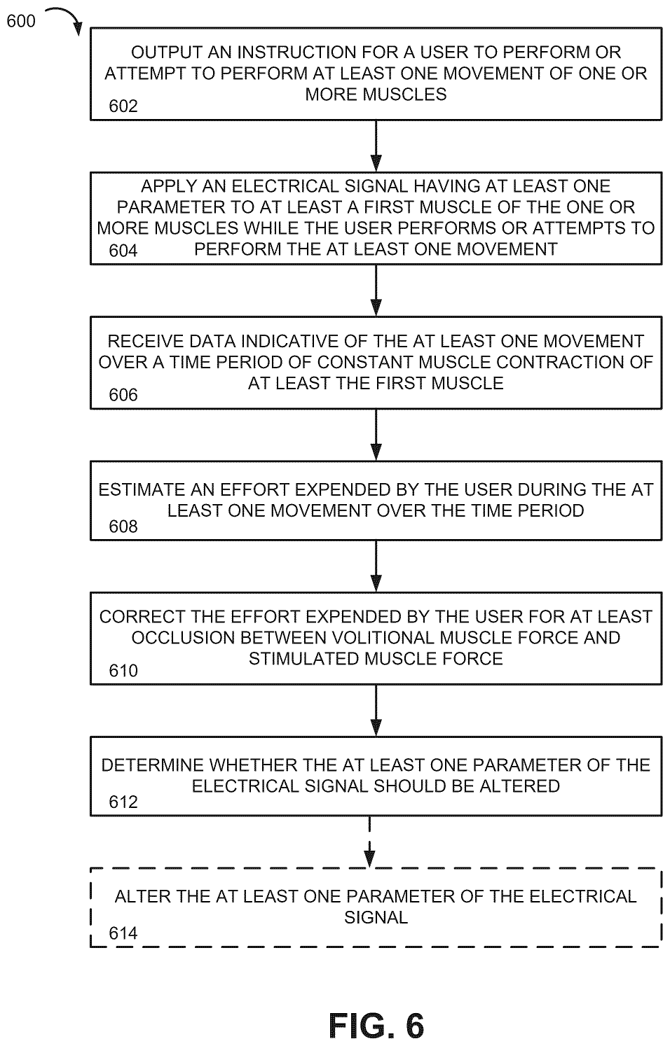

Another aspect of the present disclosure can include methods 600, 700, 800, and 900 (FIGS. 6-9) for performing assist-as-needed functional electrical stimulation (FES) (also referred to as effort dependent, closed loop FES, or the like)), or more general stimulation, while accounting for at least occlusion in the user effort measurements to reduce user slacking. The methods 600, 700, 800, and 900 can utilize a system (e.g., systems 100 or 200 shown in FIGS. 1 and 2) to deliver stimulation to assists a user in performing a movement, measure indications of movement, and re-calculate stimulation based on the measurements, occlusion, and, optionally, user fatigue. At least one step of each of the methods can be executed by at least one component that includes at least a processor (e.g., controller 16 shown in FIGS. 1 and 2 with more detail if FIGS. 3-5).

For purposes of simplicity, the methods are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods, nor are the methods necessarily limited to the illustrated aspects.

Referring now to FIG. 6, illustrated is a method 600 for performing effort dependent assist-as-needed FES to assist a user in performing at least one movement for rehabilitation and/or training purposes). At 602, a command (e.g., an instruction) for a user to perform or attempt to perform at least one movement of one or more muscles can be output (e.g., by visual display, audible instruction, or the like). The instruction can be part of a list of exercises, part of a video game, part of a routine, or the like. At 604, an electrical signal have at least one parameter can be applied to at least a first muscle of the one or more muscles (e.g., a muscle that is intended to be stimulated) and/or to at least one nerve innervating the at least the first muscle while the user performs or attempts to perform the instructed at least one movement. The electrical signal (e.g., the FES signal) can be generated by a signal generator (e.g., generator 18) and provided to at least one stimulating electrode (e.g., stimulating electrode(s) 20) for the application. The electrical signal can be generated based on parameter(s) set by the controller. The at least one parameter can include at least one of a voltage, a pulse width, a duty cycle, a frequency, a current, an amplitude, and/or a waveform change, or the like. Magnetic stimulation from at least one magnet can alternatively or additionally be applied to modulate one or more magnetic fields to stimulate the one or more muscles. At 606, data indicative of the at least one movement can be received from at least one measuring device (e.g., measuring device(s) 12) over a time period of constant muscle contraction of at least the first muscle of the one or more muscles. The data can be recorded by the at least one measuring device that can be an EMG sensor, a force sensor, a bend sensor, an inertial sensor, a motion tracking system (camera and markers), a rehabilitation device or the like. It should be noted that the at least one measuring device and the at least one stimulating electrode can be positioned on the same muscle(s) and/or different muscle(s).

At 608, an effort expended by the user during the at least one movement over the time period can be estimated. The estimated effort expended by the user can be determined based on dividing the mean absolute value of the data indicative of the movement for the time period by a maximum mean absolute value of contraction of the muscle. At 610, the estimated effort expended by the user can be corrected to account for at least occlusion between volitional muscle force measurements and stimulated muscle force measurements. An occlusion corrected effort can be determined with a linear regression breaking down volitional and stimulated efforts and an amount of occlusion. The estimated effort expended by the user can additionally and/or alternatively be corrected to account for user fatigue. For instance, a fatigue correction factor can be estimated based on a function of the median frequency of the data indicative of the movement. At 612, a determination can be made about whether the electrical signal should be altered based on the corrected effort expended by the user and a movement goal (e.g., programmed into a memory of the system). If the electrical signal should be altered, then at 614 at least one parameter of the electrical signal can be altered. In some instances, the electrical signal can be shut off if the user is determined to be too fatigued. In other instances, if the user is close to the movement goal and/or the corrected effort expended by the user is below a predetermined threshold, then the electrical stimulation can be decreased; and if the user is not close to the movement goal and/or the corrected effort expended by the user is above another predetermined threshold, then the electrical stimulation can be increased. If the electrical signal should not be altered, then the electrical signal parameter(s) can be left alone until the next determination. The method 600 can be used for rehabilitation, physical training, and/or with one or more video game systems. The user can, for instance, have a paretic side and a non-paretic side, a physical injury, a neurological condition affecting the one or more muscles, or the like or can be an athlete in training, and/or a person training to improve the performance of the one or more muscles generally. For example, a common symptom of stroke is partial paralysis of one side of the body and assist-as-needed FES can be used as part of rehabilitation plan for a user with partial paralysis of one side of the body. The assist-as-needed FES can be applied, for instance, to assist the user in re-training neurological pathways for muscle memory and improving muscle tone. The unaffected side may be left untrained, moved without the addition of stimulation, and/or used as part of the assist-as-needed FES (e.g., for contralateral control as described in more detail below).

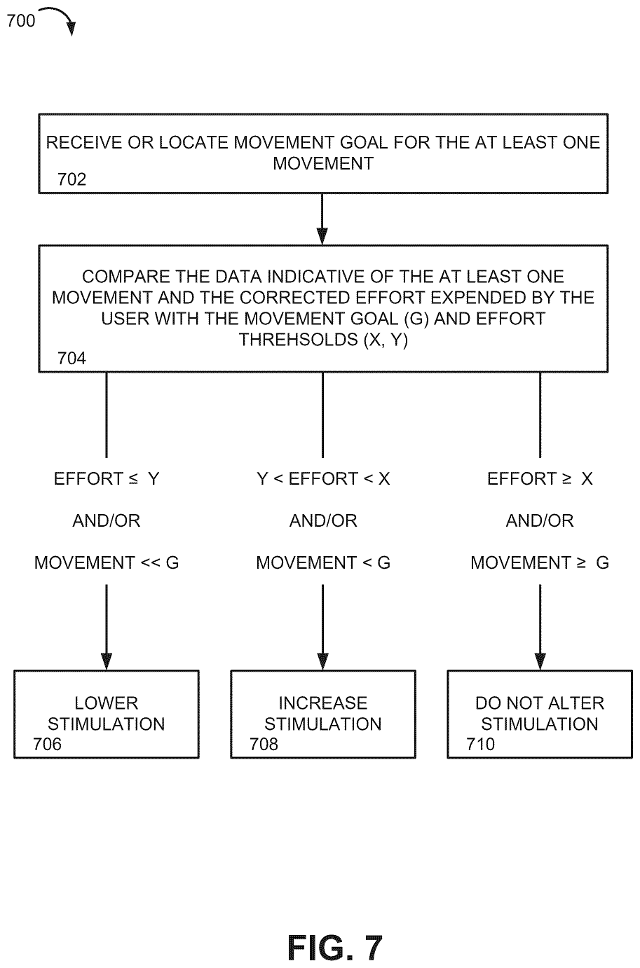

Referring now to FIG. 7, illustrated is a method 700 for determining whether and how the at least one parameter of the electrical signal should or should not be altered. At 702, a movement goal for the at least one movement can be received (e.g., from an external device 30 or input device 28) or located (e.g., in memory 22). The movement goal can be determined specifically for the user or for a population to which the user belongs. At 704, the data indicative of the at least one movement and the corrected effort expended by the user (corrected for occlusion and/or fatigue) can be compared with the movement goal (G) and effort thresholds (X, Y) where G, X, and Y are constants (e.g., can be predetermined, can be updated as the user improves, etc.). If the corrected effort expended by the user is less than or equal to a movement goal Y and/or the movement is significantly less (e.g., 60% less, 50% or less, 40% or less, or the like) than the movement goal, then, at 706, the stimulation intensity can be lowered. If the corrected effort expended by the user is between the Y threshold and the X threshold and/or the movement is only less than G (e.g., between 99% and 60%, or 50%, or 40%, or the like), then, at 708 the stimulation intensity can be increased. If the corrected effort expended by the user is greater than the upper threshold X and/or the movement at or surpassing the movement goal (G), then, at 710, the simulation can be left alone (not altered).

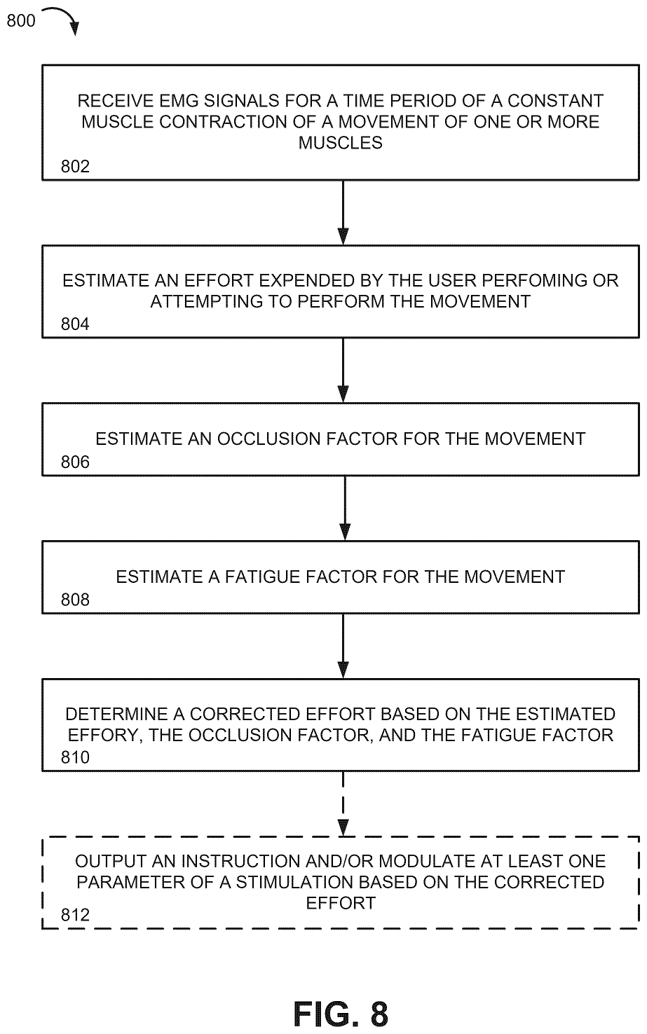

Referring now to FIG. 8, illustrated is a method 800 for estimating effort of a user of assisted functional electrical stimulation (FES) when the measuring device is at least one electromyography (EMG) electrode. At 802, EMG signals can be received for a time period of a constant muscle contraction of a movement of one or more muscles. The movement can include at least partially volitional movement (done by the user performing or attempting to perform the movement) and at least partially stimulated movement (from the FES applied by the stimulating electrode device 14). Optionally the EMG signals can be filtered to remove stimulation elicited motor responses and mean absolute value of the filtered EMG signals can be determined. At 804, the effort expended by the user performing or attempting to perform the movement can be estimated. Estimating the effort expended by the user preforming or attempting to perform the movement can include dividing the mean absolute value of the filtered EMG signals for the time period by a maximum mean absolute value of contraction of the muscle. At 806, an occlusion factor for the amount of occlusion caused by the simultaneous volitional and stimulated movement (e.g., muscle forces) can be estimated. Estimating the occlusion factor can include linearly regressing the volitional movement and stimulated movement of the user at a time in the time period. At 808, a fatigue factor for the movement can be estimated. Estimating the fatigue factor can be based on a function of the median frequency of the filtered EMG signal over the time period. And, at 810, a corrected effort can be determined based on the estimated effort, the occlusion factor, and the fatigue factor. And at 812, optionally, an instruction and/or a modulated at least one parameter of the stimulation can be output based on the determined corrected error.

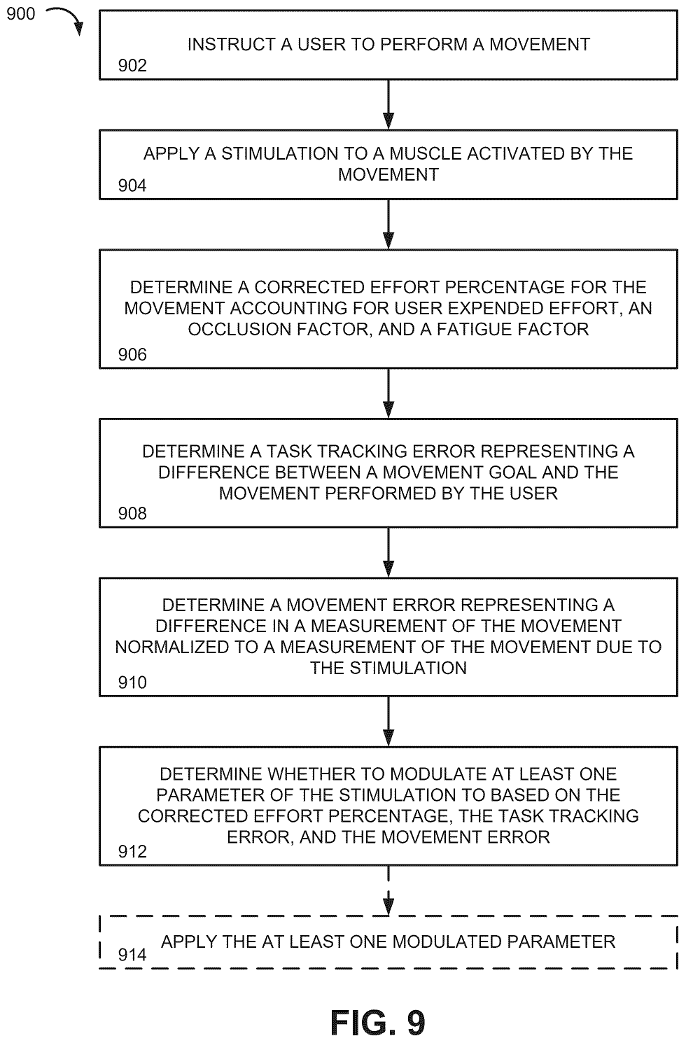

Referring now to FIG. 9, illustrated is a method 900 for controlling effort dependent contralaterally controlled FES rehabilitation. The user can be undergoing rehabilitation for stroke, for example and can have a paretic and a non-paretic side. At 902, a user can be instructed to perform a movement (e.g., of one or more muscles). The user can be instructed to perform the movement on both sides of the body (e.g., lift both right and left arms, open or close both right and left arms, or the like) and measurement device(s) can be positioned on the one or more muscles on both sides of the body. At 904, a stimulation can be applied to at least on muscle on the paretic side of the body (e.g., via a generator and at least one stimulating electrode). Measurements indicative of the movement on both sides (paretic and non-paretic) can be recorded (e.g., by EMG electrodes, force sensors, and/or the like) and received by a processor of the system (in communication with the EMG electrodes). At 906, a corrected effort percentage can be determined for the movement accounting for the user expended effort, an occlusion factor, and a fatigue factor based on the measurements indicative of the movement on the paretic side. At 908, a task tracking error representing a different between a movement goal and the movement actually performed by the user on the paretic side can be determined. At 910, a movement error representing a difference in a measurement of the movement on the paretic side normalized to a measurement of the movement due to the stimulation only can be determined. At 912, a determination about whether to modulate at least one parameter of the stimulation can be made based on the corrected effort percentage, the task tracking error, and the movement error, and the non-paretic movement. At 914, the at least one modulated parameter can optionally be applied to change the intensity of the stimulation (e.g., to prevent slacking and/or help a user working hard). For instance, the intensity of the stimulation can be increased when the corrected effort percentage is above a threshold and/or the task tracking error is below a threshold indicating the user is close to the movement goal (which can be the movement on the non-paretic side). In another instance, the intensity of the stimulation can be left alone or lowered when the corrected effort percentage is below a threshold and/or the task tracking error is above another threshold indicating the user is far from the movement goal and/or slacking. In a further instance the stimulation can be stopped, and the user can be instructed to rest when the corrected effort percentage indicates fatigue is above a threshold.

### V. EXPERIMENTAL

The following examples are shown only for the purpose of illustration and are not intended to limit the scope of the appended claims. The first experiment demonstrates that occlusion correction can significantly improve accuracy of voluntary effort estimated from EMG and that m-Wave filters can significantly affect effort estimation accuracy and the second experiment demonstrates a novel effort-dependent CCFES controller that provides assistance only when volitional hand opening is detected.

### 1. Experiment 1: Occlusion Corrected EMG-Based Effort Estimator for FES-Assisted Rehabilitation Applications

EMG-based effort estimation method was developed that accounts for FES occlusion, which is critical for improving the delivery of effort dependent FES.

### I. Methods

### A. Occlusion Corrected Effort Estimator Design

In this work, effort is defined as the percent expenditure of full capacity hand opening force such as volitional effort V=Fᵥ/Fₘₐₓ where Fᵥ is volitional force and Fₘₐₓ is the force during maximum voluntary contraction (MVC) and estimated effort is Eₑ=MAVₑ/MAVₘₐₓ, where MAVₑ is the mean absolute value of the estimated vEMG signal after m-Waves are filtered and MAVₘₐₓ is the MAV during MVC. Therefore, occlusion-corrected effort estimation is defined as

$$E = {E}_{e} + {\hat{E}}_{O} ( V , S ) , \qquad ( 1 )$$

where Ê_{O} is an estimator of occlusion factor and represents effort lost to occlusion (called occluded effort) that is added back as a correction factor, which is a function of volitional effort (V) and stimulated muscle activation intensity S (S=Fₛ/Fₘₐₓ) normalized to Fₘₐₓ in units of % Effort, where Fₛ is stimulated force. Together MAVₑ/MAVₘₐₓ is the normalized effort estimation confounded by occlusion. This occlusion correction method is designed to work at submaximal contractions and assumes a linear relationship between force and EMG MAV.

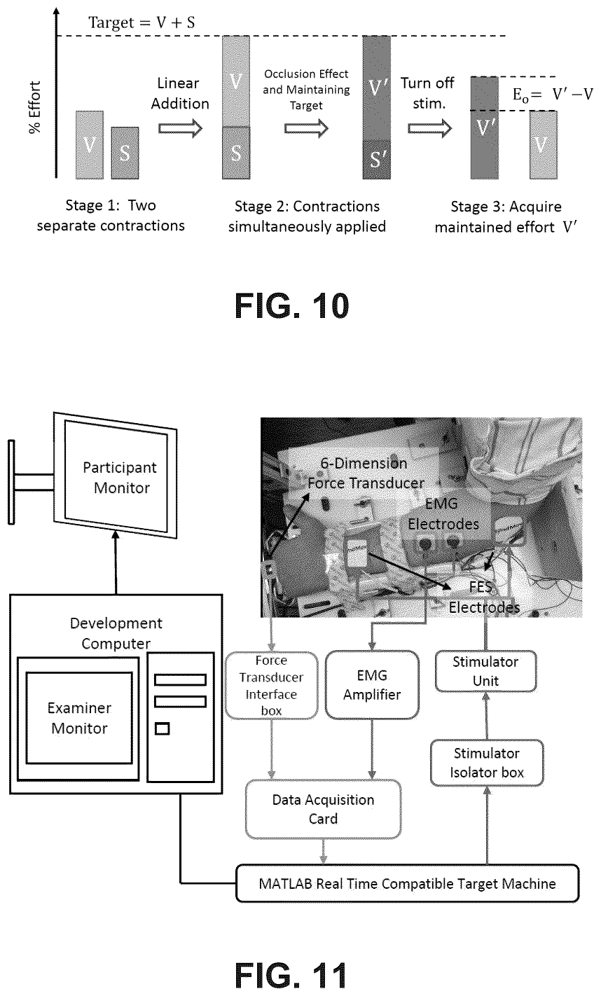

Occluded effort was estimated during different stages of the human experiment procedure using the process shown in FIG. 10. In Stage 1, V denotes a participant's volitional finger extension effort without simultaneous stimulation (referred to as ‘volitional-only’), which was normalized by a factor, Fₘₐₓ, identified at MVC. S denotes a participant's normalized stimulation-only contraction force, which is known from the recruitment curve calibration procedure. During the tracking task in the Occlusion Trials, participants were instructed to maintain a level of volitional effort that is set to Target=V+S. During the task, their finger extensor muscles are contracted by volitional effort and stimulation simultaneously. Some of the stimulated and volitional activity on motor neurons would cancel one another and result in a reduction in both effort levels due to occlusion. However, since the participants were asked to maintain the cumulative level Target, which is a sub-maximal contraction, the participants increased volitional effort to compensate for reductions in both stimulated and volitional force (see Stage 2 in FIG. 10. Changed levels for volitional effort and stimulated activation intensity are denoted by V′ and S′, consecutively. At this point, the visual feedback cursor and the stimulation were turned off and the participants were asked to maintain the volitional effort they exerted when the stimulation was on during the constant segment (see Stage 3 in FIG. 10). Using the volitional maintained level, V, occluded effort (Eₒ) for each trial can be calculated as:

$${E}_{O} = {V}^{′} - V \qquad ( 2 )$$

where, V′=F′_{V}/Fₘₐₓ, with F′_{V} being the average measured force value about 0.5 s after the stimulation and feedback are terminated. In other words V′ is the effort level participants maintained without stimulation, which we assume to be equal to the volitional effort level (on average) when the stimulation was on (see FIG. 10).

The occluded effort was calculated using equation (2) at all combinations of the voluntary effort and stimulated activation levels, and fitted the results to a linear regression to acquire an occlusion correction factor estimation model given as

$${\hat{E}}_{O} ( V , S ) = a + b V + c S \qquad ( 3 )$$

where, a, b, and c are the linear fitting coefficients.

### B. Experiments

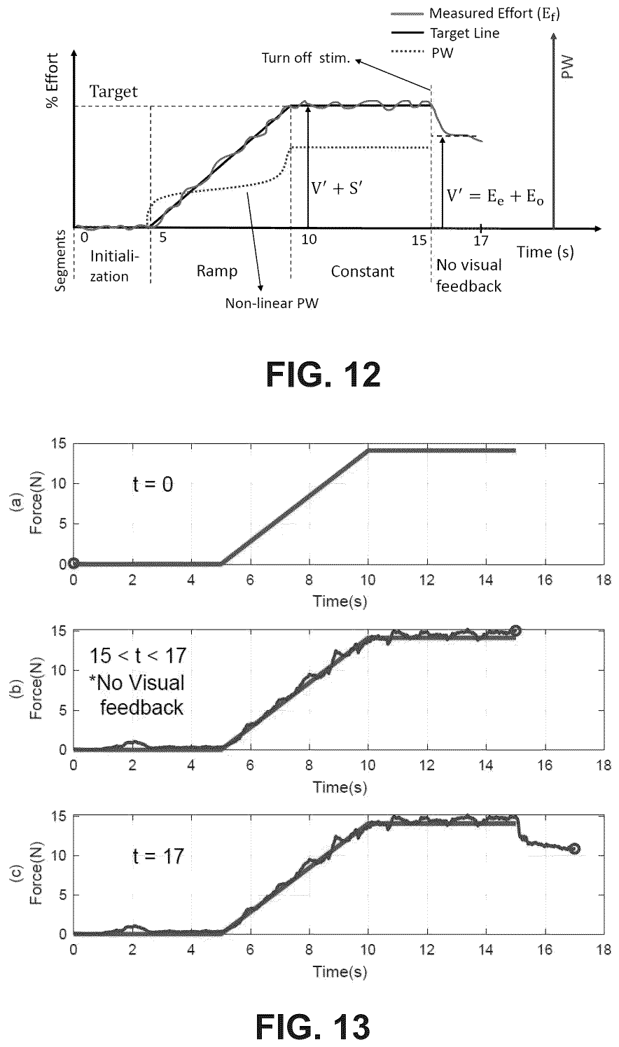

1) Subjects: Four participants with no physical disabilities (three males and one female) aged between 21 and 42 consented to participate in the following protocol, which was approved by the MetroHealth Institutional Review Board. This protocol consisted of two sessions, with the initial practice visit lasting approximately 1.5 hours, during which participants practiced force tracking tasks that were repeated at the second data collection session, which lasted about 2.5 hours.
2) Experimental Setup: Participants were seated in an upright position and placed their left hands on a custom arm support as shown in FIG. 11. Straps were utilized to limit the arm's position to the table, while plates perpendicular to the table constrained shoulder position and elbow angle. Additional straps secured the fingers to the force sensor, Nano 17 (ATI Industrial Automation Inc., Apex, NC). EMG and FES electrodes were placed on the finger extensor muscle (extensor digitorum communis) as shown in FIG. 11. Signals from EMG electrodes and force sensors were sampled at 3.5 kHz along with a trigger signal that marks the time for each stimulation pulse. Stimulation intensity was modulated by pulse width (PW), ranging between 30-150 μs, and stimulation frequency was 35 Hz. This stimulation was provided by a stimulator unit that was custom-made by the Cleveland FES Center and controlled by a Matlab Simulink target machine (Speedgoat Performance Real-Time Target Machine with IO135 DAQ) that worked in conjunction with a graphic user interface on a separate Windows PC used by study staff. A computer monitor was placed in front of the participants and displayed the desired force trajectory (as shown in the “red lines” (e.g., straight lines in elements a, b, and c) in FIG. 13) and a cursor representing the participant's real-time finger extension force (as shown in the “blue lines” (e.g., shaky lines in elements b and c and circle at 0 in element a) shown in FIG. 13).
3) Experimental Procedure: The protocol was divided into 4 parts.
(i) MVC Assessment: Participants were asked to use finger extension force to move the cursor on the display up a ramp at 6 N per second. A ramping target is used to prevent instantaneous muscle contractions. Participants were instructed to maintain the maximum force for one second once MVC was reached and the average force over this final second was defined as the MVC of each participant.
(ii) FES Recruitment Curves: Stimulation pulse width required to generate up to 30% MVC was identified for each participant and used to define the recruitment curve for the other parts of the protocol, each consisting of seven evenly distributed PW ranging from 0-30% MVC were applied for 10 seconds-long stimulation-only trials.
(iii) Occlusion trials: Participants were shown and asked to control a cursor using their finger extension force to follow the target level while stimulation was applied simultaneously at various levels (S=10, 20, and 30% MVC) of intensity. Visual feedback of the cursor was shown to the participant during segments where target level was 0% MVC, ramping, and held constant at 30% MVC, as shown in FIG. 12. During the ramp segment, the recruitment curve was inverted to apply stimulated finger extension force, and during the constant segment, the stimulation PW was kept constant. At the end of the target level, visual feedback of the cursor was turned off at the same time that stimulation was turned off and the participants were asked to maintain the same level of volitional effort that they exerted as just before stimulation ended. Participants were instructed and carefully observed by study staff to make sure they understood and successfully maintained volitional effort during a practice session before beginning the experiment session. If participants were not able to maintain the effort, the experiments were continued as normal and completed; data collected from 2 other participants was excluded from analysis and presentation of results due to inability to maintain effort. This part of the experiment was repeated for combinations of volitional activation V=10, 20, 30, and 40% MVC and stimulation levels S=0, 10, 20, and 30% MVC. This totals 48 trials, resulting from 16 conditions (4×4) repeated 3 times. The target level was set as Target=V+S. Each trial, lasting about 17 seconds, was followed by about 40 seconds of rest time to prevent muscle fatigue. An example trial where the visual feedback cursor superimposed on target level is shown in elements a, b, and c of FIG. 13 at different instances during a trial.

### C. Effort Estimation Accuracy

As illustrated in FIG. 12, maintained effort

$${V}^{′} = {E}_{e} + {E}_{O} \qquad ( 4 )$$

which is an equation equivalent of equation (1). Occlusion estimation error is defined as Error_{effort}=Ê_{O}−E_{O}; replacing Ê_{O} and E_{O} using equations (1) and (4) defines effort estimation error as:

$${Error}_{effort} = E - {V}^{′} \qquad ( 5 )$$

which also relates effort estimation E to the effort participants maintained during no-feedback segment V′.

### D. Statistical Analysis

It was tested if corrected effort levels were significantly different by comparing the uncorrected with sample size n=192 using one-way Analysis of Variance (ANOVA) assuming normal distribution. It was also tested if m-Wave filtered effort estimations are significantly different than one another and compared to unfiltered estimations using ANOVA with multiple comparisons (RStudio's Tukey HSD) with occlusion corrected effort estimation being the dependent variable and filter type (GS, comb or unfiltered) being the independent variable.

### II. Results

### A. Occlusion Estimation

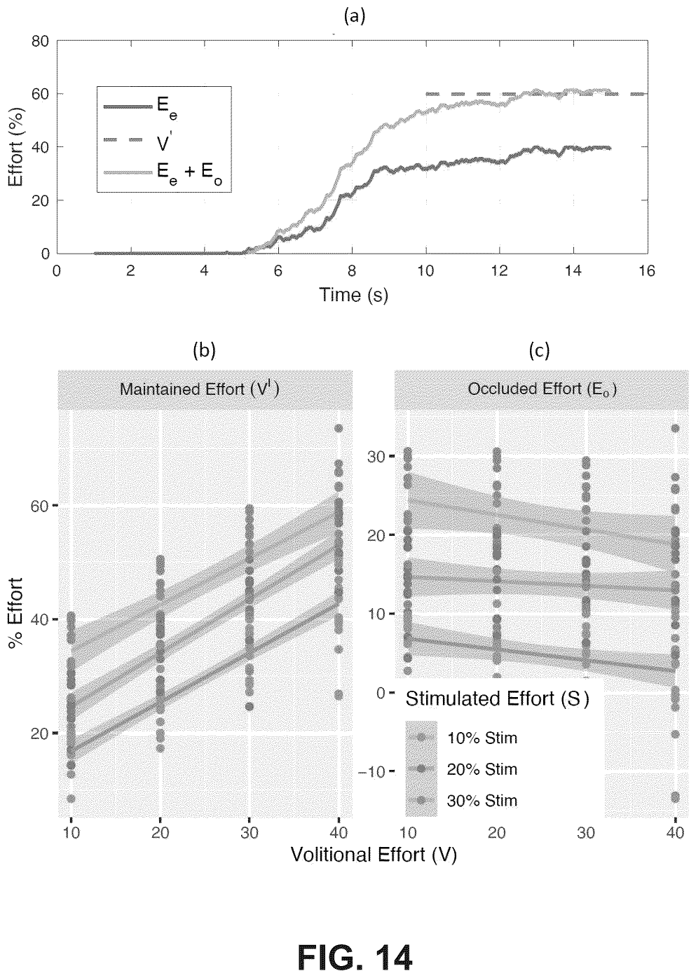

As shown in FIG. 14, element a, occlusion corrected effort is superimposed upon uncorrected effort and the average maintained effort level by participants (green dashed line). Occlusion correction improves the uncorrected effort Eₑ by making it closer to E′_{V}, the difference between the two is estimation error. Effort levels participants maintained during no feedback segment are given in FIG. 14, element a, and the occlusion estimations, calculated using equation (2) are given in FIG. 14, element b. Occlusion increases proportional to increasing stimulation and decreases proportional to volitional effort which matches previous simulations. Finally, linear regression estimations and the corresponding p-values are given in Table I (below) with b, c being statistically significant coefficients; thus Ê_{O} is a function of both simultaneously applied stimulated and voluntary levels.

TABLE 1
Linear Regression Estimations for Occlusion Estimation

| Coefficient | Estimation | p-value |
|---|---|---|
| a | −0.287 | 0.864* |
| b | −0.127 | 0.004 |
| C | 0.841 | 0.000 |

### B. Volitional Effort Estimation Accuracy

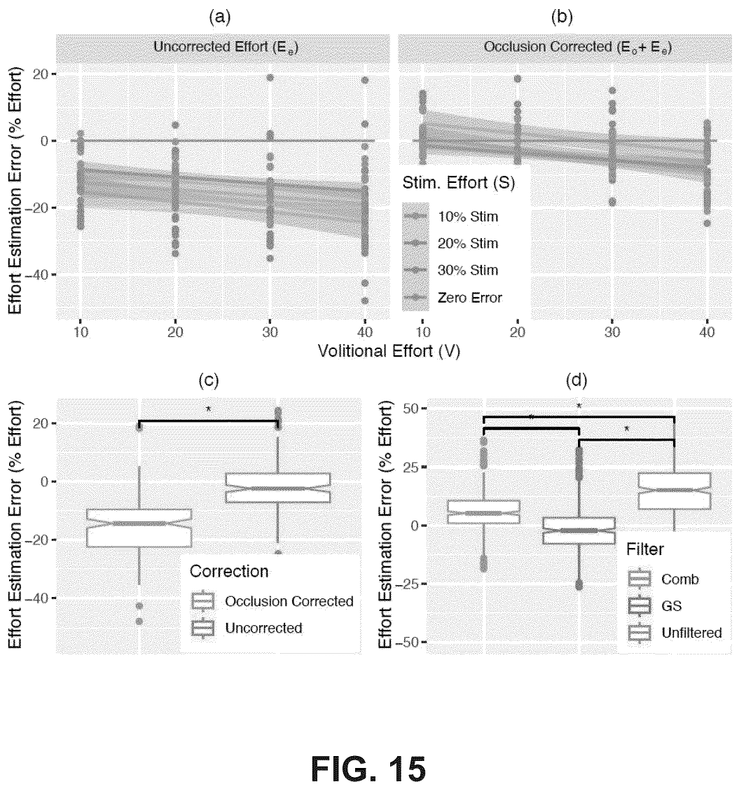

As shown in FIG. 15, element a, effort estimation error regression lines for different stimulation levels were below zero with about Error_{effort}=17% absolute effort estimation error, where the zero level represents E′ᵥ. With GS filtered estimations, occlusion correction reduced the average effort estimation error to Error_{effort}=−0.5% (average of 192 trials) effort as shown in FIG. 15, element b.

FIG. 15, element c, shows the distribution of the occlusion-corrected and uncorrected estimation errors. ANOVA results indicate that occlusion corrected effort estimation error (Error_{effort}) is significantly different (p<0.001, F(1, 191)=123.07). Multiple comparisons show that average effort estimation error was lower (p<0.001) and significantly different with a difference of about 7% for the occlusion corrected condition compared to the uncorrected effort estimation.

Finally, FIG. 15, element d, shows that effort estimation error is significantly different between comb filter, GS filter, and unfiltered (p<0.001). The GS filter performed better (average error, Error_{effort}=−1.6%) compared to comb (Error_{effort}=8.2%) and unfiltered (Error_{effort}=14.8%) estimations. ANOVA results indicate that there are significant differences (p<0.000, F(2, 191)=131.55) within groups (α=0.05).

### 2. Experiment 2: Computational Stability and Sensitivity Analysis of Effort-Dependent Contralaterally-Controlled Functional Electrical Stimulation for Hemiplegic Hand Movement Assistance

As shown, this study investigates the stability characteristics of a computational human-in-the-loop, effort dependent CCFES controller.

### 1. Methods

### A. Effort-Dependent CCFES Assumptions and System Design

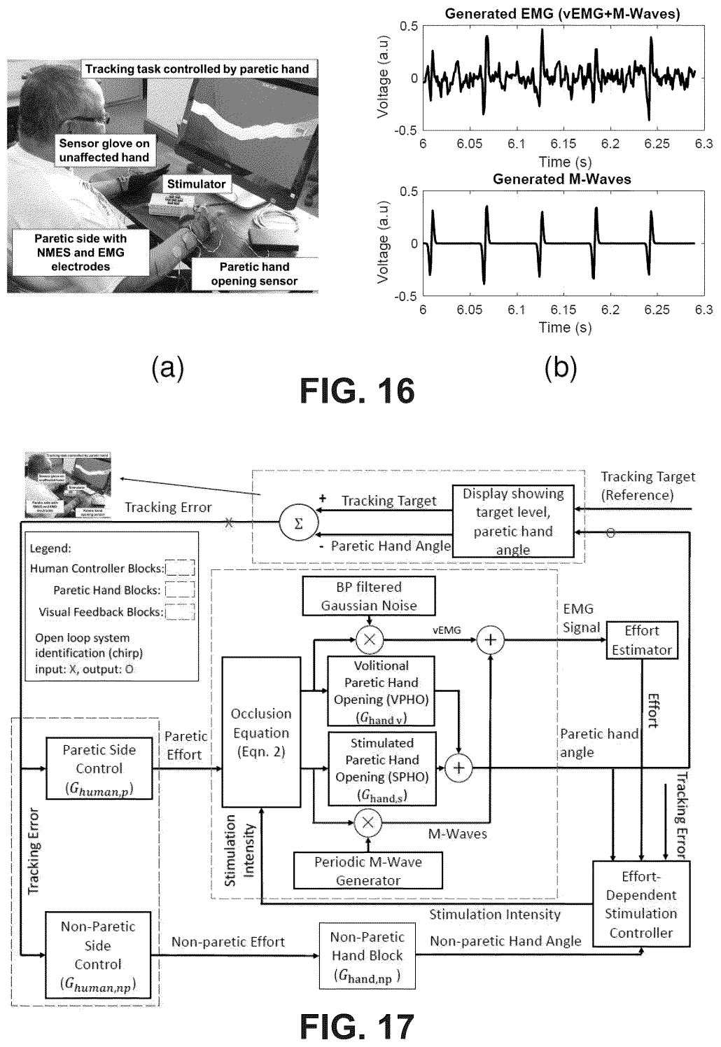

FIG. 17 shows a depiction of a block diagram of effort dependent CCFES. For an effort dependent CCFES system, it is assumed that participants wear bend sensors crossing the metacarpophalangeal joint to measure finger extension as an approximation of hand opening for both the paretic and non-paretic hands. Electrodes would be applied to the paretic extensor muscles to acquire EMG and deliver stimulation. Stimulation intensity would be a function of participant effort, hand opening angles, and tracking error to assist as needed or reduce slacking. The green dashed “Visual Feedback” block at the top of FIG. 17 is an illustration of a computer display to provide real-time visual feedback of paretic hand opening aperture superimposed on a target (which is shown in greater detail in FIG. 16, element a, while FIG. 16 element b shows a window of generated EMG signals (top) and M-waves (bottom)). The Human Controller block (indicated by red dashed lines), which models the rehabilitation participant, is continuously provided real-time feedback of their paretic hand opening and reacts to the tracking error in order to minimize it. The Paretic Hand blocks (indicated by blue dashed lines) model a paretic hand that opens slower than the non-paretic hand. To assist-as-needed, the Effort Estimator block estimates effort from the paretic side based on EMG input from the Paretic Hand Model block. The stimulation intensity law within the Effort-Dependent Stimulation Controller block governs the stimulation level while effort-dependent CCFES assists paretic hand opening as needed. Finally, the degree of paretic hand opening (as measured from the bend sensor) is fed back to the Human Controller blocks, forming a closed-loop system, simulating visual and proprioceptive feedback.

### B. Effort-Dependent CCFES Simulation

Based on FIG. 17, and the assumptions described, effort dependent CCFES was modeled in MATLAB Simulink. Both the non-paretic and paretic side hand control blocks and hand model blocks were coupled and modeled as crossover models, with the exception of additions to the paretic hand model that are described in the following subsections, both of which were driven by tracking error as illustrated in FIG. 17. The Effort Estimator block used one of three M-Wave removal filtering methods for EMG-based human effort estimation: GS, comb, or blanking. The Effort Dependent Stimulation Controller block used hand opening angles and estimated effort to calculate stimulation intensity. These blocks are explained in detail in the following subsections.
1) Paretic and Non-Paretic Hand Control Blocks: Human trajectory control behavior has been modeled as a linear controller in series with a pure delay. For this study, hand opening was modeled as a first-order low-pass filter with a cutoff frequency at 1.3 Hz, emulating volitional human motion. The crossover transfer function was defined as:

$${G}_{h u man , P} {G}_{h a n d , V} = {e}^{- s} \frac{{W}_{V}}{s} , where {G}_{h a n d , V} = \frac{1}{{T}_{V} s + 1} , \qquad ( 1 )$$

W_{V} is the crossover frequency where the magnitude line of W_{V}/s crosses the 0 dB level in the frequency response, and T is the neuromuscular transport delay. G_{human, P} is the human controller transfer function for the paretic side. G_{hand, V} is the volitional paretic hand opening (VPHO) transfer function, (frequency response of extensor digitorum communis), where T_{V}=0.5 s (baseline) is the time constant equal to 1/W_{V}. The same principle was applied to the non-paretic side. However, a smaller time constant (T_{S}=0.3 s at baseline) was used for the paretic hand to emulate slower volitional hand opening as is typical in stroke survivors.
2) Effort Estimator Block: Effort is estimated from each frame (1 stimulation period) using the adjacent-disjoint method, which is based on estimating effort using completely disjoint frames. Previously mentioned M-Wave removal filters separated vEMG from M-Waves. Following M-Wave removal, effort (E) is estimated based on the mean absolute value (MAV) feature of estimated vEMG signal for each and as

$${E}_{f} = \frac{{\overline{x}}_{f}}{max ( {\overline{x}}_{f} )} ,$$

for frame f=1:F, where F is the total number of frames, and x̅_{f} is the MAV of the sampled signal x for frame f calculated as x̅_{f}=mean(|x|ᵢ)f where xᵢ is the signal sample for i=1:n, and n is the total number samples per frame f. Thus, E is an index ranging between [0, 1], with 0 indicating no volitional effort and 1 indicating full effort.
3) Paretic and Non-Paretic Hand Model Blocks: Degree of hand opening outputted by the paretic hand model blocks is controlled by voluntary effort, V(f), and stimulus intensity, S(f), as shown by blue dashed lines (paretic hand block) as shown in FIG. 17. The model computes the occluded stimulated and volitional components, Vₒ(f), Sₒ(f) in EMG and hand opening angle, after taking into account the interactions between them, which produce EMG occlusion and hand opening occlusion as follows.

$$T ( f ) = V ( f ) + S ( f ) - k V ( f ) S ( f ) , \qquad ( 2 )$$

$${V}_{o} ( f ) = V ( f ) \frac{T ( f )}{S ( f ) + V ( f )} , {S}_{o} ( f ) = S ( f ) \frac{T ( f )}{S ( f ) + V ( f )} ,$$

Where T is total hand opening (in arbitrary units) contributed by volitional and stimulated components after occlusion, S is stimulation intensity, V is volitional effort, and k is the occlusion factor, which is a Gaussian random process whose mean value determines the average occlusion amount (e.g., k=0 for no occlusion, k=inf for complete occlusion), and the standard deviation (SD) models the variability caused by occlusion. Specifically, the mean of k was defined to be 1 and the SD of k was defined to be 0.2 (so the signal-to-noise (SNR) equals 5) for the baseline level. Vₒ and Sₒ are used for scaling vEMG, M-Wave signals, and hand opening in order to account for reductions in hand opening due to occlusion in both EMG and hand opening angle.

EMG occlusion was modeled by equation (2). The amount of reduction in vEMG MAV due to occlusion increases as stimulation intensity increases. The steady-state reduction in vEMG MAV was replicated using the formulas, assuming that occlusion of EMG MAV occurs at the same rate as occlusion in the hand opening angle. Stimulated and volitional activation components of EMG are synthesized separately and the total acquired EMG signal is a summation of M-Waves and vEMG, as shown in FIG. 17. vEMG was obtained by multiplying the voluntary component of activation, Vₒ(f), by a zero mean unity variance Gaussian noise, passed through a second-order transfer function with poles at s=625, 62.5 and a zero at s=0 (band-pass), which approximates the frequency content of real vEMG. M-Waves were modeled as the product of the stimulated component of activation, Sₒ(f), and a periodic M-Wave with 50 ms period, which is equal to the frame length of 20 Hz stimulation frequency.

The volitional component of paretic hand opening angle is assumed to be a combination of a 0.1 s delay and a first order low-pass filter with a time constant of 0.5 s. The time constant relates to the level of impairment: a greater time constant corresponds to more severely affected hand opening ability. The value selected relates to a level of impairment where volitional hand control is slower than the stimulation response of the same muscle. The transfer function, including both paretic hand control and paretic hand opening, is shown in equation (1). The stimulated component of the paretic hand opening is another first order transfer function with a time constant of 0.3 s. The total paretic hand opening angle is the sum of the stimulation and volitional muscle activation components, with each being modeled as a low pass filter as described inside the blue dashed box of FIG. 17. Similar to the paretic side, non-paretic hand opening is a combination of transport delay (0.1 s) and a first order low-pass filter with a time constant of 0.1 s. These given values are referred as baseline levels before varying them for sensitivity analysis.
4) Effort-Dependent Stimulation Controller Block: Stimulation intensity is governed by rules that are put in place to reduce slacking. If the user reduces paretic limb effort or non-paretic hand angle, then stimulation assistance is reduced. Additionally, if the tracking error eₜᵣ falls below an error threshold, c, stimulation intensity is divided by c in order to reduce slacking when it tends to occur most frequently. Hence, the control law is defined as:

$$\% Stim ( f ) = \{ \begin{matrix}R ( f ) \cdot E ( f ) , & \vert {e}_{tr} ( f ) \vert \geq c \\ R ( f ) \cdot R ( f ) \cdot \frac{{e}_{tr} ( f )}{c} , & \vert {e}_{tr} ( f ) \vert < c\end{matrix} , \qquad ( 3 )$$

where normalized stimulation ranges from [0, 1], with 0 indicating no stimulation and 1 indicating the stimulation intensity that causes full paretic hand opening. As mentioned previously, E(f) represents estimated effort, sampled at a frame f, R represents non-paretic hand opening percentage with 0% indicating hand fully closed and 100% indicating maximum hand opening angle. eₜᵣ is the difference between the tracking target and paretic hand opening.

### C. Linearization and Stability Analysis

System components that include nonlinear terms, such as the absolute value used to calculate MAV, and the terms that are difficult to model as linear transfer functions, such as GS filter, were linearized using system identification via optimization. The stability analysis was performed using the identified transfer function with tracking error as input and paretic hand angle as output (X and O symbols in FIG. 2). Using the linearized transfer functions and their uncertainty range, stability was analyzed by the Nyquist method. Finally, stability analysis was conducted by statistically comparing system performance metrics across a range of system parameter variations. The details of this process are given in the following subsections.
1) Linearization: The stimulation controller (3) was linearized around the operating point (50% paretic hand opening), the tracking error was assumed to be less than the threshold |eₜᵣ(f)|<c as in equation (3) and the non-paretic hand was at 50% opening (R was defined as 0.5) at steady state. The following steps were taken to further linearize effort dependent CCFES.
a) Simulated nonlinear system frequency response: A chirp signal with a frequency range of 0-10 Hz was applied to the open-loop input of paretic side and non-paretic side because most human motion fits within that frequency range, and the continuous open-loop output signal, paretic hand angle, and the input signal (as shown in FIG. 17) were sampled within Simulink at 20 Hz. A different random number seed for the Gaussian random processes was used for each experiment condition combination to approximate physiologic variation. Open loop system identification was performed on the system shown in FIG. 17 with visual feedback elements removed (which are shown as the visual feedback block of FIG. 17) so that it is compatible with Nyquist stability analysis. The control model shown in FIG. 17 was simulated in MATLAB Simulink 10.2 with time step set to 0.1 ms. The outcome data were used for identifying the open-loop frequency responses.

Hand opening is not a linear process and the intrinsic properties of muscles, such as viscosity and stiffness, vary with respect to hand kinematics but no closed form nonlinear mathematical models have been defined in the literature. System-level analyses were conducted before using linearized models such as the case of people with stroke and Parkinson's disease. Therefore, a conventional first step is to evaluate system stability linearized about an operating point, where viscosity and stiffness variations are limited. The muscle stiffness was estimated as 6-7.5 N·m·rad⁻¹, and the viscosity estimation was between 0.02-0.03 N·m·s·rad⁻¹ but the estimations varied based on the experimental conditions such as muscle activation and velocity. We also foresee that the impact of the nonlinearities of human muscle dynamics would be dominated by linear effects of the human motor controller, which tends to reduce the combined system and human dynamics to an integrator. Therefore, we assume that variations in intrinsic properties of the muscles are limited compared to the other components such as human controllers and EMG signals.
b) System identification via optimization: Using least square estimation via MATLAB's fmincon.m, the open-loop frequency response of effort-dependent CCFES, L(z), was fitted to the transfer function L_{fit}(z), where z=e^{jw}. L(z) was calculated as L(z)=Y(z)/U(z), where Y(z) is the frequency response in the open-loop output signal (paretic hand opening percentage shown in FIG. 17) and U(z) is the frequency response of the input, chirp signal (shown as tracking error in FIG. 17). The fitted transfer function is a sampled version of an integrator multiplied by a sampled first order transfer function, and is defined as:

$${L}_{fit} ( z ) = \frac{{x}_{1}}{{z}^{2} - ( {x}_{2} + 1 ) z + {x}_{2}} , \qquad ( 4 )$$

where x₁ and x₂ are the fitting parameters.

### D. Sensitivity Analysis

Sensitivity analysis can reveal system performance characteristics prior to collection of clinical data and is conducted for two reasons. First, simulation parameters can change due to factors such as hand impairment severity, muscle stiffness, variance of the EMG signal, hand opening speed, and changing linearized parameters due to nonlinearities. Second, estimations in literature may become imprecise once real-world conditions differ from those used to measure the parameters.

Sensitivity analyses were conducted on Nyquist stability margins and performance measures by incrementally varying one of the parameters within a range, simulating the system while holding all the other parameters at their baseline values, and repeating the process again until the range of the parameter value was covered. A total of 12,000 computational simulation trials were performed, which is the result of 1,200 model parameter conditions (three filter types, 4 parameters, 100 steps across the variation range of each parameter), with each condition repeated 10 times using different random seeds in order to account for the variability that occurs in repeated trials. The simulations occurred with tracking target set to 50% hand opening and time duration of the simulation set to 10 s to reflect the duration of tracking activities during therapy. In the sensitivity analyses, the value for each parameter was varied from half to two times the baseline values.
1) Defining Uncertainty Bounds and Stability Analysis: Uncertainty bounds of L_{fit}(z) were defined according to an additive perturbation model. The uncertainty range is defined by the weighting function W. Where W(z) was |W(z)|=|L(z)−L_{fit}(z)|. The stability margins were investigated via the Nyquist method, which maps the “D” contour using the nominal transfer function L_{fit}(z). The system is closed-loop stable if the Nyquist contour of the open loop transfer function L_{fit}(z) does not encircle the critical point ‘−1’. The magnitude, |W(z)|, defines the radius of the uncertainty circles around the Nyquist contours, thus the range of uncertainty at each frequency ω. The distance from the critical point to the closest circle perimeter is known as the “sensitivity” of the transfer function. A stability margin greater than 1 is considered highly stable.
2) Focus on simulating two types of variability sources: those related to hand opening and EMG signal. Stimulated and volitional hand opening models are transfer functions of hand opening (see FIG. 17).
a) Volitional paretic hand opening (VPHO): VPHO is associated with the level of impairment of the user and will be referred to as “impairment level”. The associated simulation parameter is the time constant τᵥ of the VPHO transfer function located in the paretic hand blocks of FIG. 17. It is important to note that VPHO is not a measure of impairment, which is a far more complicated term that cannot be modeled as a transfer function but is a measure of the impairment's impact on hand opening velocity. Larger values of tauᵥ indicate a more impaired (slower) hand opening response.
b) Stimulated paretic hand opening (SPHO): Similar to VPHO, stimulation response was modeled as the time constant, τₛ, of the SPHO transfer function. Smaller time constant results in faster stimulation response.
c) EMG Occlusion: Average occlusion was adjusted by setting the occlusion factor's mean value (k from equation (2)) to 1.0 in our paretic hand opening model. Larger k values would result in more occlusion, reducing the level of hand opening and EMG amplitude due to simultaneously applied stimulated and volitional activity; k=0 would, similarly, mean no occlusion. In order to model for occlusion variability, a standard deviation (SD) was introduced into the occlusion factor, k. Thus, the sensitivity analysis was run by varying the occlusion factor's SD between 0.1 to 0.4.
d) Temporal changes between M-Waves (M-Wave phase shift): The M-Waves occur following each stimulation pulse with a delay called M-Wave phase shift. However, the delay varies slightly with each stimulation pulse. This variation is called temporal variance of the M-Waves. The proposed model accounted for the level of variation in the M-Wave phase shift by changing the SD of M-Wave phase shift.
3) Case Scenarios: For greater clinical relevance, the stability and performance characteristics of effort-dependent CCFES were demonstrated with hypothetical participants and a hypothetical M-Wave removal filter model that is designed to emulate removal of a percentage of the M-Waves.

Effort dependent CCFES was simulated with the following three different participant models of volitional and stimulated paretic hand opening response (see Table II below), which are intended to model some behavior (e.g., hand opening velocity) related to varying levels of impairment or spasticity. Participant scenario A features slow volitional paretic hand opening with fast stimulated opening, which could be presented by a potential participant whose volitional hand opening is severely impaired with minimal spasticity. Participant scenario B features fast volitional paretic hand opening with slow stimulated opening, which could be presented by a potential participant whose volitional hand opening velocity is not so severely impaired but presents symptoms of spasticity. Participant scenario C features slow volitional hand opening with slow stimulated opening, which could be presented by a potential participant whose volitional hand opening velocity and stimulation response is severely affected. It is important to note that our models are not meant to physiologically mimic stroke impairment or spasticity, but they model some of the hand opening behavior which could result from varying clinical conditions.

Case A simulates a better-performing M-Wave removal filter by attenuating the amplitude of each M-Wave by 80% (referred to as M=80% in Table II). Similarly, Case B simulates a poorly-performing M-Wave removal filter by attenuating the amplitude of each M-wave by 20% (referred to as M=20%). Case C simulates participant slacking by introducing a slacking term to the human controller when the hand opening parameters are at their baseline values. The input of the human controller block, u (effort), decayed as an exponential function where u=uₒ·e^{-t}/T_{f} with a time constant T_{f}=10 s when the tracking error is small.
4) Performance Measures:

Tracking Error (eₜᵣ) is defined as the absolute value of the difference between the reference position and the model's position output (representing the degree of paretic hand opening). Tracking SNR is calculated as the mean value of paretic hand position divided by its SD for tracking a constant target position. R-Squared value was acquired by fitting the estimated effort and actual effort sample sets to a linear regression line. Estimated Effort SNR measures the resolution of effort estimation. It is calculated as the mean value of the estimated effort divided by its SD. Nyquist Stability Margin was used to determine how sensitive the system stability is with respect to the changes in parameters.

TABLE II
Stability Margins of Hypothetical Participants with Different M-
Wave Removal Filters and of Hypothetical Filtering Quality

| Case | | | Nyquist Stability |
|---|---|---|---|
| Scenarios | Description | Parameter | Margins |
| Case A | Slow Volitional, | Tᵥ = 0.7 s | Comb: 0.974 |
| | Fast Stimulated | Tₛ = 0.2 s | GS: 0.983 |
| | Hand Opening | | Blanking: 0.978 |
| Case B | Fast Volitional, | Tᵥ = 0.3 s | Comb: 0.978 |
| | Slow Stimulated | Tₛ = 0.5 s | GS: 0.982 |
| | Hand Opening | | Blanking: 0.979 |
| Case C | Slow Stimulated | Tᵥ = 0.7 s | Comb: 0.986 |
| | Hand Opening | Tₛ = 0.5 s | GS: 0.976 |
| | | | Blanking: 0.969 |
| Case A | Better Filter | M = 80 percent | Hypothetical: 0.97 |
| | Performance | | |
| Case B | Poor Filter | M = 20 percent | Hypothetical: 0.969 |
| | Performance | | |
| Case C | Slacking | T_{f} = 10 s | Comb: 0.977 |
| | | | GS: 0.968 |
| | | | Blanking 0.982 |

5) Statistical Analysis: The primary hypothesis of system stability was evaluated using descriptive statistics of the stability margin to determine the level of stability based on conventional categories.

For the secondary goal of characterizing sensitivity, the magnitude of changes of the performance measures and stability margins across the parameter variability ranges were investigated using a 3-way Analysis of Variance (ANOVA) with interactions (dependent variable: magnitude of change, independent variables: performance measures, parameter variability, and filter type). Post-hoc multiple comparisons with Bonferroni correction were used to examine interactions to test if the performance measure, parameter variability, and filter type have a significant effect on the magnitude of change. The term “magnitude of change” refers to absolute difference between the minimum and maximum performance measures over the variability range of one parameter. Magnitude of change was used to assess sensitivity, with a low magnitude of change indicating low sensitivity (ideal because low sensitivity indicates a minimal chance of having lower performance metrics and stability margins with respect to varying clinical conditions). High magnitude of change indicates high sensitivity, which is caused by large changes in performance measures with respect to parameter variation (not ideal for effort dependent CCFES).

### III. Results

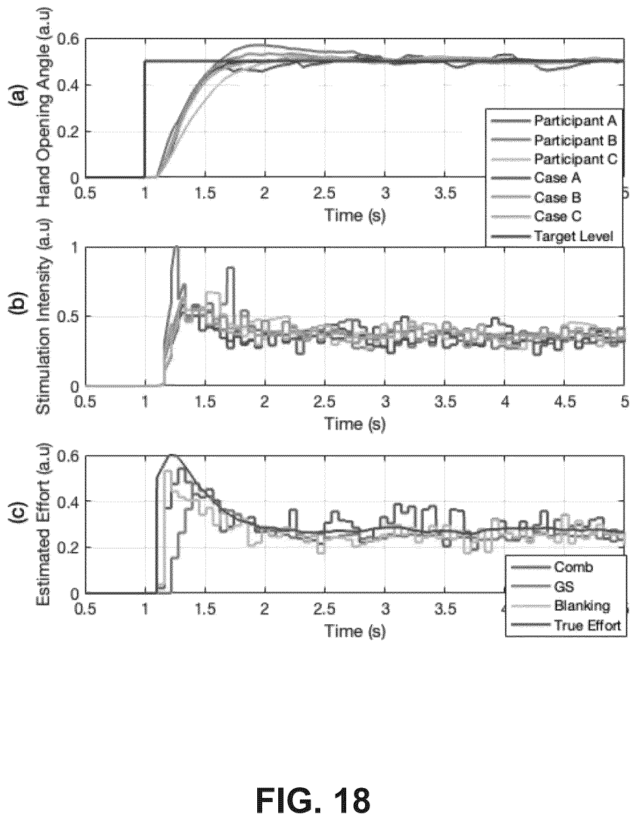

FIG. 3 shows hand opening and effort estimation signals of a representative time-domain simulation of a CCFES user attempting and maintaining a target goal of 50% hand opening (denoted as “paretic hand angle” in FIG. 17) using effort dependent CCFES with baseline model parameter values. The target level in FIG. 18, element a is superimposed on paretic hand opening with different M-Wave removal filters. FIG. 18, element c, shows volitional effort estimations by three M-Wave removal filters superimposed to estimate simulated volitional effort.

### A. System Linearization

The coefficients of equation (4) for the open-loop system with a chirp input for each of the three M-Wave filters varied between 0.0067-0.0075 for x1 and 0.5242-0.5082 for x2. In terms of the crossover model, the crossover frequencies for all three linearizations are approximately 0.3 Hz.

The Nyquist contours of the magnitude response were found to be far away from the coordinates of (−1, 0), which is the critical point for stability. The Nyquist contours of the three M-Wave filters at their baseline stability margins are very close to one another: 0.971 for blanking, 0.967 for GS filter, and 0.967 for comb filter.

### B. Case Scenarios

All the hypothetical Participants A, B, and C were able to maintain target tracking as revealed in FIG. 18, element a, and were stable, as evidenced by the corresponding Nyquist margins for each filter type given in Table II. The simulated hypothetical filters that simulated poor and good filtering quality scenarios, Case A and Case B, were also stabilized by the human controller with their target tracking given in FIG. 18, element a, and the stability margins are given in Table II. Stimulation levels were also stable with the stimulation intensity variations, as shown in FIG. 18, element b, being below maximum possible stimulation level, 1 a.u.

### C. Sensitivity Analysis

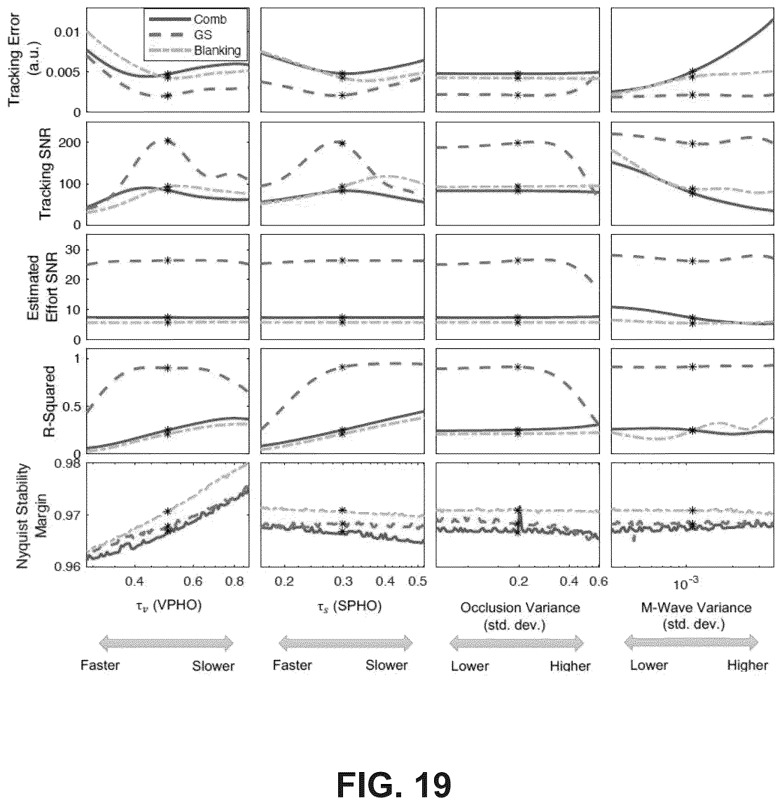

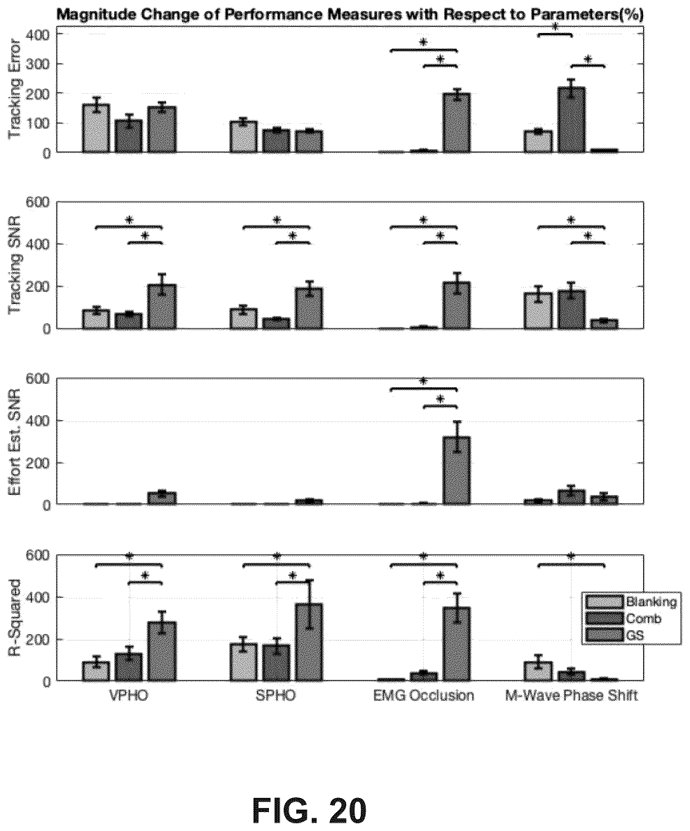

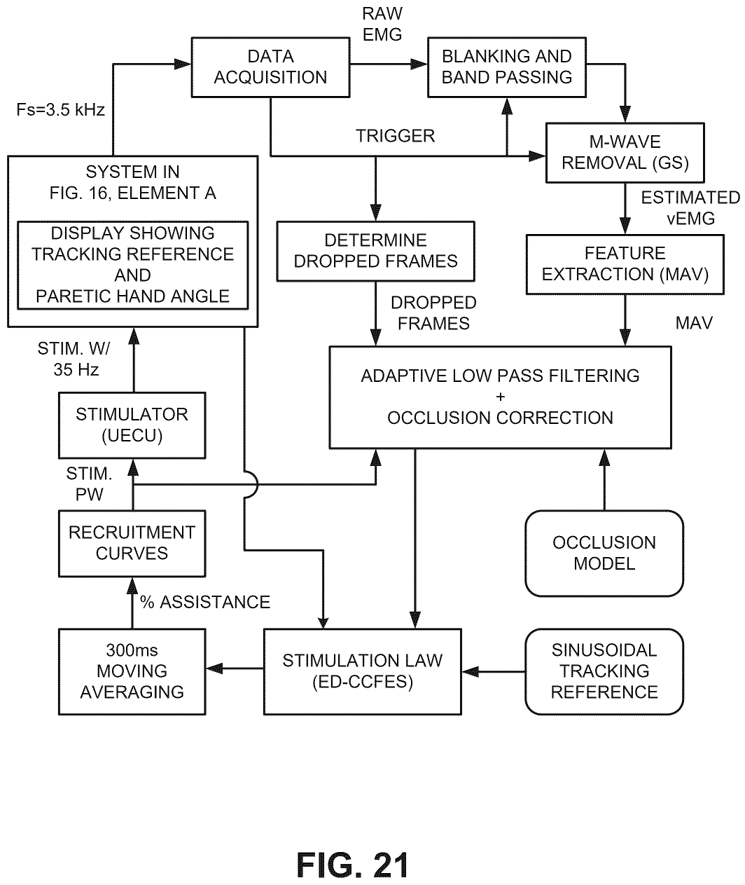

The sensitivity of system performance measures is plotted in FIGS. 19 and 20. FIG. 19 shows the average (of 10 runs each with different random seeds and the same model parameters) performance measures and Nyquist stability margins as a function of parameter values. The combination of the mean and SD of magnitude of change, difference between the minimum and the maximum values of the sensitivity lines given in FIG. 19, for Nyquist stability margin were less than 2.2% (Table III). FIG. 20 shows the magnitude of change along with any notable post-hoc multiple comparisons (α=0.05) of the significant 3-way interaction between performance measure*parameter variability*filter type (p<0.01, F (24, 599)=11.94).

TABLE III
Magnitude of Change for Nyquist Stability Margins %

| | | | EMG | M-Wave |
|---|---|---|---|---|
| Filter Type | VPHO | SPHO | Occlusion | Variance |
| Comb | 1.6 ± 0.06 | 0.5 ± 0.26 | 0.42 ± 0.14 | 0.21 ± 0.03 |
| GS | 1.8 ± 0.03 | 0.15 ± 0.04 | 0.58 ± 0.04 | 0.02 ± 0.04 |
| Blanking | 2.1 ± 0.07 | 0.23 ± 0.03 | 0.15 ± 0.06 | 0.16 ± 0.05 |

### 3. Experiment 3: Implementing a Stimulation Control Law That Aims to Reduce Slacking

As shown, this study investigates the implementation of a computational human-in-the-loop, effort dependent CCFES controller that assists as needed for post-strike hand rehabilitation. The study proposes an effort-dependent (ED) CCFES system that estimates volitional effort in real-time and adjusts stimulation intensity accordingly, thereby preventing slacking and maximizing patient engagement. The fundamental hypothesis was that participants would exhibit higher voluntary effort under ED-CCFES paradigm compared to conventional, effort independent CCFES.

### I. Aim

To test this hypothesis, a comprehensive approach was undertaken that included: (1) the development of a pipeline for real-time EMG acquisition and processing, which involves filtering and artifact removal to account for M-waves and frame drops; (2) implementation of an adaptive low-pass filter and occlusion-correction algorithm to accurately estimate volitional EMG; and (3) integration of these estimates into a stimulation controller that adjusts assistance based on the user's effort. The ED-CCFES controller was then be evaluated through a series of sinusoidal tracking tasks in participants with chronic stroke, comparing the outcomes of stimulation-only, volitional-only, and combined effort-dependent versus effort-independent stimulation trials in a randomized order.

Through these steps, this experiment enhanced the state of FES-based rehabilitation by addressing the critical limitation of slacking and adopting “assist-as-needed” principles from robotic rehabilitation in an FES context. The intention was not only to provide more effective rehabilitative interventions for individuals recovering form stroke, but also to establish a foundation for the wider integration of effort-dependent algorithms into future FES technologies.

### II. Methods

### A. Stimulation Controller

The stimulation Controller is designed to mimic the adaptive intervention strategies employed by occupational therapists (OTs) during stroke rehabilitation. Occupational therapists typically monitor a patient's effort and provide assistance only when necessary. They dynamically adjust their assistance based on the patient's tracking performance and volitional engagement, preventing slacking while maintaining the “just-right challenge” to promote motor relearning [73]. Similarly, the stimulation controller adapts electrical stimulation in real-time, addressing the key limitations of traditional CCFES systems by incorporating effort estimation, paretic and non-paretic hand angles, and tracking error. This warrants that stimulation is only provided when needed and at appropriate levels, promoting active engagement.

Stimulation intensity is calculated as follows:

$${S}_{i} = \{ \begin{matrix}{θ}_{np , i} \cdot {E}_{i} , & {e}_{tr , i} > c and {Error}_{hand} \leq h \\ {θ}_{np , i} \cdot {E}_{i} \cdot \frac{\vert {e}_{tr , i} \vert}{c} , & 0 \leq {e}_{tr , i} \leq c and {Error}_{hand} \leq h \\ 0 , & {e}_{tr , i} < 0 or {Error}_{hand} > h\end{matrix} , \qquad ( 1 )$$

Where E is the participant's estimated effort, θ_{np,i} is the non-paretic hand angle, e_{tr,i} is the tracking error, c is the tracking threshold, Error_{hand} is the difference between two normalized hand angles defined as Error_{hand,i}=|θ_{np,i}−θ_{p,i}|, threshold h is the hand error threshold, Sᵢ is the stimulation intensity, and i is the frame indice.

In this controller, each term plays a role in emulating a therapist's interventions. The term θₙₚ represents the angle of the non-paretic hand and serves as the primary input for controlling stimulation. In CCFES systems, the movement of the non-paretic hand is used to guide the stimulation of the paretic hand. This mirrors the strategy used by CCFES therapy, where the unaffected limb is often engaged to facilitate movement in the paretic limb. Similarly, estimated effort (E) makes sure that stimulation intensity is directly proportional to the patient's active volitional effort. This is a similar strategy adopted by OTs encourage patients to attempt movements independently before providing assistance, the law increases stimulation only if the effort is detected while the tracking error (eₜᵣ) is high. This is motivated by a premise that participants might disengage if they sense that the system will provide adequate stimulation assistance regardless of how much they personally contribute. Finally, the threshold, h, ensures adherence to the conventional CCFES procedures, which encourages participants to open both hand hands synchronously to trigger contralateral motor pathways.

### B. ED-CCFES Design

Closed-loop neuromuscular stimulation and tracking system designed to assist paretic hand movements through FES, namely ED-CCFES, is depicted in FIG. 21. At its core, the system acquires raw surface EMG signals from the user's paretic and non-paretic hands, processes these signals to extract meaningful features, and then adjusts the intensity of the stimulation. The goal is to enable the user to track a sinusoidal reference target shown on the display. FES is delivered at 35 Hz to optimize the recruitment of muscle activation while EMG is and other signals are sample at 3.5 KHz to ensure adequate resolution. The intervening blocks between the delivery of stimulation and data acquisition underscores the complexity of the rehabilitation engineering beneath and is discussed in the following.

In FIG. 21, “Data Acquisition” block acquires surface EMG signal in real time, and immediately following acquisition, a blanking and band passing process (20-500 Hz) is employed to remove the high-frequency content that is not relevant to the nature of surface EMG signal [28], and to blank the stimulation artifacts. Because FES pulses can induce large artifacts known as M-waves, the “M-Wave Removal (GS)” block is triggered after blanking to isolate and remove the stimulation response.

The “Adaptive Low Pass Filtering” and “Occlusion Correction” stages refine the EMG-derived effort estimation. Low-pass filtering is performed to reduce high-frequency fluctuations in EMG features, namely MAV. At the same time, occlusion model represents the correction coefficients that is used to correct the effort estimation to account for EMG occlusion. The output of this block forms the “Effort Estimation” input before slacking preventing control strategy.

The ED-CCFES approach then calculates the appropriate stimulation intensity based on the user's effort estimation, non-paretic and paretic hand angles, and tracking reference. Before stimulation level is finalized, it passes through the “300 ms Moving Averaging” block to dampen abrupt transitions in stimulation, for reducing unwanted fluctuations and gradually adapting to the needed force. The “Recruitment Curves” module determines how much current or pulse width (PW) is needed to elicit a certain percentage of muscle recruitment. These curves are especially helpful for balancing the non-paretic and paretic hand angles, ensuring safe and progressive rehabilitation.

Finally, the system's primary objective is fulfilled through the “Sinusoidal Tracking Reference.” While the display shows a target trajectory, it simultaneously offers real-time feedback on the user's actual hand movements, guiding them to open and close their paretic hand in sync with the time-varying reference angle. This coupling effectively closes the loop and fosters relearning as well as neuromuscular adaptation. By integrating EMG processing, effort estimation, adaptive filtering, and precisely tuned stimulation control, the system can bolster the user's voluntary effort. The slacking-reducing, effort-dependent assist-as-needed real-time controller has the potential to foster meaningful neuroplastic changes, which can lead to more impactful rehabilitation outcomes.

### C. Experiments Goals and Data Analysis

The overarching objective of the experimental design is to examine whether ED-CCFES controller can reduce “slacking,” i.e., whether participants with stroke remain more actively engaged in their rehabilitation when stimulation parameters are tailored to their voluntary effort. Before the main experiment begins, individuals with stroke undergo a calibration phase to derive “recruitment curves” and “occlusion model.” This personalized calibration is particularly important due to variability in neuromuscular responses among different individuals and variability of occlusion models depending on various factors.

Once calibration was complete, participants perform a sinusoidal tracking task in which they open and close both their paretic and non-paretic hands to follow a reference trajectory displayed on a screen. Four main task conditions were tested:
(1) Stimulation-only tracking: participants are instructed to only exert effort on their non-paretic side and relax on paretic hand, and the paretic hand movement is predominantly driven by electrical stimulation.
(2) Volitional-only tracking: requires the participant to move the paretic side along their active range without any stimulation assistance.
(3) CCFES: participants are instructed exert effort on both sides to practice the tracking tasks on their paretic side. Here, stimulation intensity was determined proportional to the non-paretic hand angle.
(4) ED-CCFES: participants are instructed to exert effort on both sides; however, stimulation intensity is delivered based on effort with c=0.25 and h=0.35

Following the voluntary and stimulation-only trials (repeated 5 times each), CCFES and ED-CCFES are presented in a randomized order, which is also repeated five times each, yielding 20 total trials. This experiment was tested on participants (1 time with S7 and two times with S1) whose population profile is given in the Table 1.

TABLE 1
Population profile of the participants

| Test | Participant | Age | Sex | Affected Side | FMA |
|---|---|---|---|---|---|
| 1 | S1 | 55 | M | Right | 9 |
| 2 | S7 | 61 | F | Left | 11 |
| 3 | S1 | 55 | M | Right | 9 |

### III. Results/Discussion

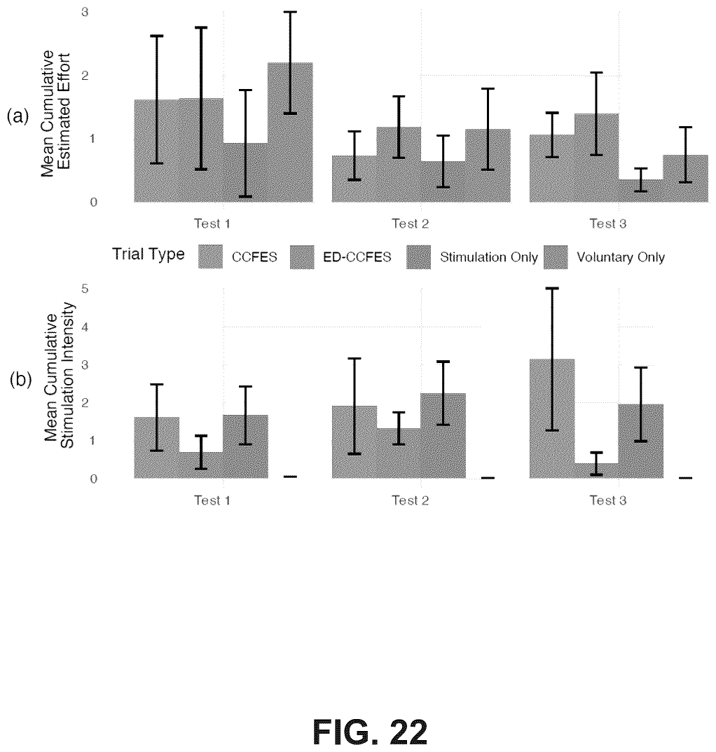

The results (shown in FIG. 22) indicate that participants using the ED-CCFES system maintained effort levels higher than those observed with conventional CCFES, particularly in Tests 2-3. This finding supports the hypothesis that people who practiced hand opening with ED-CCFES exerted more effort compared to conventional CCFES. The results also show that ED-CCFES uses less stimulation intensity compared to conventional CCFES in certain trials, which might suggest increased exerted effort for the same tracking task. This reduction in stimulation may have clinical advantages, including minimizing participant discomfort, avoiding potential muscle fatigue from prolonged stimulation, and extending the battery life of portable FES devices. Importantly, reduced stimulation levels with maintained or increased voluntary effort suggest that ED-CCFES supports task performance with a lighter reliance on external assistance, a desirable characteristic for fostering long-term recovery.

From the above description, those skilled in the art will perceive improvements, changes, and modifications. Such improvements, changes and modifications are within the skill of one in the art and are intended to be covered by the appended claims.

The following is claimed:

1. A system comprising:
at least one measuring device configured to record data indicative of at least one movement of one or more muscles a user;
at least one stimulating electrode configured to provide an electrical signal to at least a first muscle of the one or more muscles; and
a controller, in communication with the at least one measuring device and the at least one stimulating electrode, the controller comprising:
a non-transitory memory configured to store instructions, and
a processor configured to execute the instructions to:
output an instruction for the user to perform or attempt to perform the at least one movement of the one or more muscles, wherein in response to the instruction, the user performs or attempts to perform the at least one movement of the one or more muscles that makes a volitional muscle force in the one or more muscles,
apply, via the at least one stimulating electrode, the electrical signal having at least one parameter to the at least the first muscle of the user during the user performing or attempting to perform the at least one movement of the one or more muscles, wherein the electrical signal causes a stimulated muscle force in the one or more muscles,
receive, from the at least one measuring device, the data indicative of the at least one movement of the one or more muscles over a time period of constant muscle contraction, wherein the data indicative of the at least one movement of the one or more muscles includes both the stimulated muscle force and the volitional muscle force,
estimate an effort expended by the user during the at least one movement of the one or more muscles over the time period,
correct the effort expended by the user for at least occlusion between the volitional muscle force and the stimulated muscle force,
determine whether the electrical signal should be altered based on the corrected effort expended by the user and a movement goal,
if the electrical signal should be altered, then change the at least one parameter of the electrical signal, and
if the electrical signal should not be altered, then make no change to the electrical signal.

2. The system of claim 1, wherein the at least one measuring device comprises at least one electromyography EMG electrode, at least one force sensor, at least one bend sensor, at least one inertial sensor, at least one motion capture system, and/or at least one rehabilitation device.

3. The system of claim 1, wherein the at least one measuring device and the at least one stimulating electrode are on a same muscle of the one or more muscles.

4. The system of claim 1, wherein the instructions further comprise correct the effort expended by the user for muscle fatigue based on a fatigue correction factor.

5. The system of claim 4, wherein the instructions further comprise estimating the fatigue correction factor based on a function of the median frequency of the data received from the at least one measuring device.

6. The system of claim 1, wherein the electrical stimulation is used for functional electrical stimulation and the functional electrical stimulation is applied for rehabilitation of a limb comprising the one or more muscles.

7. The system of claim 1, further comprising a display, audio, and/or tactile device configured to convey the instructions to perform or attempt to perform the movement of the one or more muscles to the user and/or alert the user if the effort expended by the user is below a predetermined threshold.

8. The system of claim 1, wherein the determine whether the electrical signal should be altered based on the corrected effort expended by the user and the movement goal, further comprises determining the user's proximity to the movement goal,
wherein if the user is close to the movement goal and/or the corrected effort expended by the user is below a predetermined threshold, then the electrical stimulation should be decreased, and
wherein if the user is not close to the movement goal and/or the corrected effort expended by the user is above another predetermined threshold, then the electrical stimulation should be increased.

9. The system of claim 1, wherein the system is configured to prevent and/or reduce slacking in a user for rehabilitation purposes.

10. The system of claim 1, wherein that at least one parameter is at least one of voltage, pulse width, duty cycle, frequency, current, amplitude, or waveform change.

11. The system of claim 1, further comprising at least one magnet, wherein the at least one magnet is configured to modulate one or more magnetic fields to stimulate the one or more muscles of the user.

12. The system of claim 1, wherein the system is configured for rehabilitation of the user, physical training of the user, and/or use with one or more video game systems.

13. A method for estimating effort of a user of assisted functional electrical stimulation (FES), comprising:
receiving, by a system comprising a processor, electromyography (EMG) signals for a time period of a constant muscle contraction of a movement, wherein the movement is at least partially driven by volitional movement and at least partially driven by stimulated movement from the FES;
estimating, by the system, effort expended by the user performing or attempting to perform the movement;
estimating, by the system, an occlusion factor for the amount of occlusion caused by the volitional movement and the stimulated movement simultaneously;
estimating, by the system, a fatigue factor for the movement; and
determining, by the system, a corrected effort based on the estimated effort, the occlusion factor, and the fatigue factor.

14. The method of 13, further comprising:
filtering, by the system, the EMG signals to remove stimulation elicited motor response artifacts; and
determining, determining by the system, a mean absolute value of the filtered EMG signals.

15. The method of 14, wherein estimating the effort expended by the user preforming or attempting to perform the movement comprises dividing the mean absolute value of the filtered EMG signals for the time period by a maximum mean absolute value of contraction of the muscle.

16. The method of 13, wherein estimating the occlusion factor comprises linearly regressing the volitional movement and stimulated movement of the user at a time in the time period.

17. The method of 13, wherein the estimating the fatigue factor is based on a function of the median frequency of the filtered EMG signal over the time period.

18. The method of 13, further comprising modulating at least one parameter of the electrical stimulation in response to the corrected effort determination.

19. A method for controlling effort dependent contralaterally controlled functional electrical stimulation (FES) rehabilitation comprising:
instructing, by a system comprising a processor, a user to perform a movement;
applying, by the system, a stimulation comprising a FES signal to a muscle activated by the movement;
determining, by the system, a corrected effort percentage for the movement that accounts for user expended effort, an occlusion factor, and a fatigue factor;
determining, by the system, a task tracking error representative of the difference between a movement goal and the movement of the user;
determining, by the system, a movement error representative of a difference in a measurement of the movement normalized to a measurement of the movement due to the stimulation;
determining, by the system, whether to modulate at least one parameter of the stimulation based on the corrected effort percentage, the task tracking error, and the movement error, wherein the at least one parameter of the stimulation controls stimulation intensity; and
applying, by the system, the at least one modulated parameter to a stimulator to modulate the stimulation.

20. The method of claim 19, further comprising increasing the intensity of the stimulation when the corrected effort percentage is above a threshold and/or the task tracking error is below a threshold indicating the user is close to the movement goal.

21. The method of claim 19, further comprising not changing the intensity of the stimulation or lowering the intensity of the stimulation when the corrected effort percentage is below a threshold and/or the task tracking error is above another threshold indicating the user is far from the movement goal.

22. The method of claim 19, further comprising stopping the stimulation and instructing the user to rest when the corrected effort percentage indicates fatigue is above a threshold.
